(19) United States
(12) Patent Application Publication
(10) Pub. No.: US 2007/0196502 A1
(43) Pub. Date: Aug. 23, 2007

(54) Flowable particulates

(75) Inventors: Paul R. Mort III, Cincinnati, OH (US); Nigel Patrick Somerville Roberts, Newcastle (GB); Mark Edward Wandstrat, Cleves, OH (US); George Gregory Spontak, Loveland, OH (US); Julie Ellis, Newcastle (GB); John Demetrius Hatjopoulos, Cincinnati, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/788,099

(22) Filed: Apr. 19, 2007

Related U.S. Application Data
(63) Continuation-in-part of application No. 11/407,617, filed on Apr. 20, 2006.
Application No. 11/407,617 is a continuation-in-part of application No. 11/012,755, filed on Dec. 15, 2004.
(60) Provisional application No. 60/793,352, filed on Apr. 20, 2006.
(60) Provisional application No. 60/831,494, filed on Jul. 18, 2006.
(60) Provisional application No. 60/544,666, filed on Feb. 13, 2004.

(51) Int. Cl. A61K 9/28; A61K 9/50

(52) U.S. Cl. 424/490; 426/635; 427/2.14

(57) ABSTRACT

The present invention relates to particles, compositions comprising said particles, and processes for making and using the aforementioned particles and compositions. When employed in compositions or alone such particles provide controlled dosing and improved performance without the negatives that are associated with fluid products.

### CROSS REFERENCES TO RELATED APPLICATION

This application claims priority under 35 U.S.C. §119(e) to U.S. Provisional Application Ser. No. 60/793,352 filed Apr. 20, 2006, and U.S. Provisional Application Ser. No. 60/831,494 filed Jul. 18, 2006, and is a continuation-in-part of and claims priority under 35 U.S.C. §120 to U.S. patent application Ser. No. 11/407,617 filed Apr. 20, 2006, which in turn is a continuation-in-part of and claims priority under 35 U.S.C. §120 to U.S. patent application Ser. No. 11/012,755 filed Dec. 15, 2004, which in turn claims priority under 35 U.S.C. §119(e) to U.S. Provisional Application Ser. No. 60/544,666 filed Feb. 13, 2004.

### FIELD OF INVENTION

This invention relates to flowable particulates and compositions comprising such particulates; and processes for making and using such particulates and products.

### BACKGROUND OF THE INVENTION

Flowability is a desirable characteristic for most products as it provides an ease of dispensability that can permit accurate, controlled dosing. Solid products do not provide the steady rate of pouring or discharge of product in a narrow bulk flow stream, especially when the width of the stream is narrow compared to a product's particle size. As solid products do not provide the degree of flowability that is desired, products typically take the form of fluids, particularly liquids. Unfortunately, such fluids require complex dosing equipment or they are messy as they can drip after dosing and thus contaminate surfaces, such as the container opening or associated dosing device. Furthermore, such contamination can make reopening the container difficult as the product can glue the container's opening device to the body of the container. In addition, dosing of liquids from a container, such as a rigid container, requires ingress of vapor to fill the volume displaced by outflow of the liquid. Thus if dosing is performed through a narrow egress, an additional ingress port may be required.

Thus, while particulates are disclosed, see for example WO 2006/048142 A2; WO 2007/014601 A1 and U.S. Pat. No. 5,324,649, what is needed is a particulate that flows in a manner that is similar to a fluid, yet which does not have the disadvantages of a fluid. The particle taught herein satisfies such need.

### SUMMARY OF THE INVENTION

This invention relates to flow able particulates comprising certain particles and compositions comprising such particulates; and processes for making and using such particulates and products.

### DETAILED DESCRIPTION OF THE INVENTION

Definitions

As used herein, the term “cleaning composition” includes, unless otherwise indicated, granular or powder-form all-purpose or “heavy-duty” washing agents, especially cleaning detergents; hand dishwashing agents or light duty dishwashing agents, especially those of the high-foaming type; machine dishwashing agents; mouthwashes, denture cleaners, car or carpet shampoos, bathroom cleaners; hair shampoos and hair-rinses; shower gels and foam baths and metal cleaners; as well as cleaning auxiliaries such as bleach additives or pre-treat types.

As used herein, the articles “a” and “an” when used in a claim, are understood to mean one or more of what is claimed or described.

As used herein, the term “layer” means a partial or complete coating of a layering material built up on a particle's surface or on a coating covering at least a portion of said surface.

As used herein, the term “Product Growth Factor” means the ratio of the product mass to the mass of the initial seeds.

As used herein, the term “Layering Rate” is defined as:
Layering Rate=M_{product}/(M_{seed}*t_{layering})
where M_{product} is the total product mass; M_{seed} is the total initial seed mass; and t_{layering} is the layering material application time. In the case of a batch process, t_{layering} is the elapsed time of layering including binder and layering powder additions. In the case of a continuous process, t_{layering} is the total product rate divided by the total mass holdup of material in the layering process unit operation.

As used herein, the term “Product Yield” means the ratio of the net product mass to the total product mass. The net product mass is determined after post-layering treatments such as but not limited to drying, elutriation, and classification. The total product mass is the product mass after layering but before post-layering treatment.

As used herein, the term “Yield Rate” means the multiplicative product of the Layering Rate and the Product Yield: Yield Rate=(Product Yield)*(Layering Rate).

As used herein, the term “seed” means any particle that can be coated or partially-coated by a layer. Thus, a “seed” may consist of an initial seed particle or a seed with any number of previous layers.

As used herein, the term “Critical Gap Dimension” means the diameter of the largest circle that can be fully inscribed within the open flat planar orifice area perpendicular to the product flow direction through said orifice.

As used herein the term “independent streams” means that said streams are physically separated and/or separated in time. In one example, independent streams refers to separate feed streams of binder and layering powder that are added at the same time, but in spatially separate locations within the mixing process. In another example, a mixing process with one or more ingress locations is used, and binder and layering powder are added into the process at different times.

As used herein the term “swept volume” means the volume that is intersected by a mixing tool attached to a rotating shaft during a full rotation of the shaft.

As used herein the term “hydratable material” means a solid material that is capable of reacting with water or a composition containing water to form a solid hydrate material.

It is understood that the test methods that are disclosed in the Test Methods Section of the present application must be used to determine the respective values of the parameters of Applicants' inventions as such inventions are described and claimed herein.

Unless otherwise noted, all component or composition levels are in reference to the active level of that component or composition, and are exclusive of impurities, for example, residual solvents or by-products, which may be present in commercially available sources.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Particulate

The particulates disclosed herein can provide controlled dosing without the negatives that are associated with fluid products. As the benefits of flowability are desired in many products, in one aspect said particulate may be an industrial chemical; edible food, instant beverage mix, drug or nutriceutical; a pet food and/or pet care particulate; or a detergent, fabric treatment, personal cleaning, hair care and/or fertilizer particulate. Versions of Applicants' particulates may be used in any application, particularly wherein flowability is desired, for example, cleaning and/or treatment products, industrial chemicals, fertilizers, pharmaceuticals, foods, pet-foods, instant beverages, and nutraceuticals.

In one aspect, Applicants' particulate has a Relative Jamming Onset of from about 2 to about 14, from about 2.5 to about 12, from about 3 to about 10, or even from about 4 to about 8 particles. In another aspect, Applicants' particulate has a median particle size of from about 250 microns to about 4,000 microns, from about 300 microns to about 1,200 microns, from about 400 microns to about 1000 microns, from about 500 microns to about 850 microns, or even from about 600 microns to about 750 microns. In another aspect, Applicants' particulate has a size distribution span of from about 1.0 to about 1.75, from about 1.05 to about 1.6, from about 1.1 to about 1.45, or even from about 1.1 to about 1.3. In another aspect, Applicant's particulate has a bulk density of from about 350 grams/liter to about 2000 grams/liter, from about 500 grams/liter to about 1200 grams/liter, from about 600 grams/liter to about 1100 grams/liter, or even from about 700 grams/liter to about 1000 grams/liter. In another aspect, Applicant's particulate has a median particle aspect ratio of from about 1.0 to about 1.4, from about 1.05 to about 1.3 or even about 1.1 to about 1.25. In one aspect, Applicants' particulate may comprise particles that comprise a seed and a layer said layer at least partially coating said seed. In one aspect, Applicants' particulate may comprise particles that comprise a seed and a layer comprising a binder and a layering powder, said layer at least partially coating said seed. In another aspect, Applicants' particulate may comprise particles that comprise a plurality of seeds, by way of a non-limiting example, 1, 2, 3, 4, 5, 6, 7, 8, 9, or even 10 seeds. In another aspect, Applicants particulate may comprise particles that comprise a plurality of discrete layers, by way of a non-limiting example, 1, 2, 3, 4, 5, 6, 7, 8, 9, or even 10 layers. In another aspect, Applicants particulate may comprise particles that comprise a plurality of binder materials, by way of a non-limiting example, 1, 2, 3, 4, 5, 6, 7, 8, 9, or even 10 binder materials. In another aspect, Applicants particulate may comprise particles that comprise a plurality of layering powders, by way of a non-limiting example, 1, 2, 3, 4, 5, 6, 7, 8, 9, or even 10 layering powders. In one aspect, the binder of Applicants' particulate may comprise an oil, for example, a perfume oil, nutritional oil and/or a flavor oil.

In one aspect, Applicant's particulate contains both acid and alkaline materials. In one aspect, Applicant's particulate effervesces on contact with water.

Suitable materials for making the aforementioned particle depend on the end product application. Such materials are known to the skilled artisan. However they may include, for example, seed materials, binder materials and layering powder materials—each of the aforementioned materials may be an active material or an inert material.

Seed materials are commonly available as granular grades of feedstock materials. Said feed stocks may be raw materials obtained from a supplier or may be an intermediate granule that is produced by any number of granulation processes. Suitable seeds may have a median particle diameter of from about 150 microns to about 1700 microns, from about 200 microns to about 1200 microns, from about 250 microns to about 850 microns, or even from about 300 microns to about 600 microns; a seed bulk density of from about 50 grams per liter to about 2000 grams per liter, from about 200 grams per liter to about 1650 grams per liter, from about 350 to about 1200 grams per liter or even from about 400 grams per liter to about 850 grams per liter; optionally a size distribution span of from about 1.0 to about 2.0, from about 1.05 to about 1.7, or even from about 1.1 to about 1.5; and optionally a median particle aspect ratio of from about 1 to about 2, from about 1 to about 1.5, or even from about 1 to about 1.3. For detergent applications, suitable active seed materials include, but are not limited to, materials selected from the group consisting of surfactants, builders, buffering agents, soluble polymers, optical brighteners, and mixtures thereof. In certain applications, an active oil-based component may be mixed in molten carrier such as tristearin or wax and then prilled to form a solid seed. Stabilizers, antioxidants, and preservatives may be incorporated within active seeds. Suitable inert seed materials include, but are not limited to, materials selected from the group consisting of salts, bi-salts, starches, sugars and mixtures thereof. In one aspect, porous seeds may be used as a carrier for other active materials, including but not limited to, perfumes, flavors, vitamins, nutritional oils, and microencapsulates thereof. In one aspect, said active is not a surfactant. In one aspect, hollow particles may be used as seeds. In one aspect, an encapsulate may be used as a seed, said encapsulate comprising a wall that encapsulates a material, such as a perfume, flavor, vitamin, nutritional oil and mixtures thereof.

The seeds disclosed in the present specification may have any combination of median particle diameter, seed bulk density, size distribution span, median particle aspect ratio and type and number of components detailed above and throughout this application, including the claims and examples.

Suitable active binder materials include, but are not limited to, materials selected from the group consisting of acid surfactant precursors, surfactants, polymer solutions or their acid precursors, silicones, chelant solutions, silicate solutions, cellulosic solutions or dispersions, dye solutions, pigment dispersions, molten polymers, molten waxes, molten fatty acids, nutritional oils and mixtures thereof. Suitable inert binder materials include, but are not limited to, materials selected from the group consisting of water, salt solutions, sugar solutions and mixtures thereof. Suitable binders may include, but are not limited to, solutions, dispersions or emulsions of actives in an active or inert base. Examples of actives include, but are not limited to, oil solubles such as mixed tocopherols, BHT, gallates, ubiquinone, fatty esters of ascobic acid, beta carotene, and polyphenols. Suitable binders may have a viscosity of from about 0.5 cp to about 4000 cp, from about 1 cp to about 2000 cp, from about 2 cp to about 1000 cp, from about 5 cp to about 600 cp, or even from about 20 cp to about 400 cp. While not being bound by theory, it is believed that suitable binders may function in Applicants' process by first wetting the surface of seed particles, rendering the seed particles sufficiently sticky to bind the layering powder onto the seed structure, and then most preferably undergoing a chemical or physical transition from a liquid to a solid or semi-solid phase. In one aspect, the liquid binder may transform to a solid phase by a chemical reaction with the layering powder. In one aspect, a molar excess of the layering powder reactant is required in order to achieve substantially complete conversion of the binder reactant. In one aspect, the liquid binder may transform to a solid phase by solidification on cooling from a hot melt. In one aspect, a reactive liquid binder may be first blended with a molten binder, and then the blended binder system is transformed into a solid phase by a combination of chemical reaction with layering powder and congealing upon cooling, thus reducing the excess amount of layering powder reactant that may be required with the reactive binder alone. In one aspect, a liquid binder may transform to a solid phase by a chemical reaction with another binder composition. In one aspect, a liquid binder may transform to a solid phase by evaporation of a solvent. In one aspect, the binder may comprise a liquid.

Suitable active layering powder materials include, but are not limited to, materials selected from the group consisting of surfactants, soluble polymers, builders, buffering agents, starches, optical brighteners, dyes, pigments and mixtures thereof. Suitable inert layering powder materials include, but are not limited to, materials selected from the group consisting of salts, bi-salts, sugars, starches, polymers, pigments, dyes and mixtures thereof. Other actives, stabilizers, preservatives or antioxidants can be incorporated into the dry layering powder, including ascorbic acid, erythorbic acid, the fatty acid esters of ascorbic acid, bisulfites, pyrophosphates, tetrasodium hydryoxyethylidene diphosphonate (HEDP), trisodium ethylenediamine-disuccinate (EDDS), chelants, for example citric acid, tetrasodium carboxylatomethyl-glutamate (Dissolvine® or GLDA), trisodium methylglycinediacetate (Trilon® M or MGDA), diethylene triamine pentaacetic acid (DTPA) and ethylenediamine tetraacetic acid (EDTA), and herbal extracts, for example, rosemary extract. In one aspect, layering powder compositions contain at lease one hydratable material. Suitable layering powders may have a median particle size from about 1 micron to about 100 microns, from about 2 to about 50 microns or even from about 3 microns to about 30 microns. In one aspect of Applicants' invention, a dry solids comminution mill may be used to reduce the particle size of the layering materials to the desired particle size. A suitable comminution mill can be obtained from Hosokawa Alpine Aktiengesellschaft & Co. OHG, Augsburg, Germany; Netzsch-Feinmahltechnik GmbH, Selb/Bayern, Germany; RSG Incorporated, Sylacauga, Ala., USA. In one aspect, small-scale prototypes may be used. For example, a bench-top micronizer may be used to reduce the particle size of layering powders; a suitable bench-top micronizer is available from Retsch GmbH, Haan, Germany

In one aspect, the particle's seed may comprise an active material and at least one of the layers coating said seed comprise an active material, for example an active binder, an active layering powder or mixture thereof. In another aspect, the particle may comprise an inert seed and at least one of the layers coating said seed may comprise an active material, for example an active binder, an active layering powder or mixture thereof. In another aspect, the particle may comprise a seed that may comprise an active material and one or more inert layers.

In one aspect, the particle's active ingredients may include hygroscopic materials.

In another said aspect, said hygroscopic materials are located in the seed or inner layer structure, with an outer layer consisting of comparatively less hygroscopic or non-hygroscopic materials. In one aspect, applicant's particulate has a Rapid Stability Relative Jamming Onset of from about 2 to about 18, from about 2 to about 14, from about 2.5 to about 12, from about 3 to about 10, or even from about 4 to about 8 particles.

Depending on the application, suitable materials for the seed, binder, and/or layering powder may be obtained from a variety of suppliers. For selected applications including detergent and cleaning formulations, foods, pet-foods, pharmaceuticals, nutriceuticals, and agricultural chemicals, materials can be obtained from Innophos, Incorporated of Cranbury, N.J., USA; Rhodia of Paris, France; FMC Corporation of Philadelphia, Pa., U.S.A.; Jost Chemicals of St. Louis, Mo., U.S.A.; General Chemical Corporation of Parsippany, N.J., U.S.A.; Ulrich Chemicals of Indianapolis, Ind., U.S.A.; Jones-Hamilton Company of Walbridge, Ohio, U.S.A.; Sigma Aldrich Corporation of St. Louis, Mo., U.S.A., Cargill Incorporated of Minneapolis, Minn., U.S.A.; International Ingredient Corporation of St. Louis, Mo., U.S.A.; National Starch Corporation, Bridgewater, N.J., U.S.A.; PQ Corporation of Philadelphia, Pa., U.S.A.; BASF of Ludwigshafen, Germany; Dow Chemical Company of Midland, Mich., U.S.A.; Hercules Incorporated of Wilmington, Del., U.S.A.; Shell Chemical LP of Houston, Tex., U.S.A.; Procter & Gamble Chemicals of Cincinnati, Ohio, U.S.A.; Rohm and Hass Company of Philadelphia, Pa., U.S.A.; Akzo Nobel, Arnhem, NL; Ciba Specialty Chemicals Corporation of Newport, Del., U.S.A.; Clariant Corporation of Charlotte, N.C., U.S.A.; and Milliken Chemical Company of Spartanburg, S.C., U.S.A.

The particulates disclosed in the present specification may have any combination of Relative Jamming Onset, median particle size, size distribution span, bulk density, median particle aspect ratio and type and number of components detailed above and through out this application, including the claims and examples.

Process of Making Particles

The particles of the present invention and/or other particles may be made as follows:

In one aspect, particles may be made by contacting a particle and a binder comprising a liquid in a counter-rotating dual-axis paddle mixer, wherein said axes are oriented horizontally with paddles attached to the counter-rotating axes and said binder is introduced into said mixer through an ingress located at the bottom of said dual-axis paddle mixer.

In one aspect, said counter-rotating dual-axis paddle mixer has a converging flow zone located in between the counter-rotating paddle axes. In one aspect, the swept volumes of said counter-rotating paddle axes overlap within the converging flow zone. In one aspect, the swept volumes of said counter-rotating paddle axes do not overlap within the converging flow zone. In one aspect, there is a gap in the converging flow zone between the swept volumes of said counter-rotating paddle axes.

In one aspect, said binder is introduced into said counter-rotating dual-axis paddle mixer such that said binder is directed upward into the converging flow zone between the counter-rotating paddle axes. In one aspect said counter-rotating dual-axis paddle mixer has a converging flow zone between the counter-rotating paddle axes and the swept volumes of said counter-rotating paddle axes do not overlap within the converging flow zone and said binder is directed into the gap between the swept volumes of said counter-rotating paddle axes.

In one aspect, said binder has a viscosity of from about 1 cp to about 100000 cp, from about 20 cp to about 10000 cp, from about 50 cp to about 5000 cp, or even from about 100 cp to about 2000 cp.

In one aspect, said ingress comprises a distributor pipe located below the converging flow zone of the counter-rotating paddle axes said distributor pipe comprising one or more holes.

The particle disclosed in the present application may also be made via the teachings and examples disclosed herein. While only a single mixing unit may be required, multiple mixers may be employed, for example cascading mixers of progressively increasing volume capacity. In any of the aforementioned aspects of the invention, the binder may comprise a liquid.

In one aspect, the particles disclosed herein may be produced by a process comprising:
a.) layering a mass of seeds, said seeds having:
   (i) a median particle diameter of from about 150 microns to about 1700 microns, from about 200 microns to about 1200 microns, from about 250 microns to about 850 microns or even from about 300 microns to about 600 microns;
   (ii) optionally a size distribution span of from about 1.0 to about 2.0, from about 1.05 to about 1.7, or even from about 1.1 to about 1.5;
   (iii) a seed bulk density of from about 50 grams per liter to about 2000 grams per liter, from about 200 grams per liter to about 1650 grams per liter, from about 350 to about 1200 grams per liter or even from about 400 grams per liter to about 850 grams per liter; and
   (iv) optionally a median particle aspect ratio of from about 1 to about 2, from about 1 to about 1.5, or even from about 1 to about 1.3;
said layering process comprising independently contacting said mass of seeds with a liquid binder and a layering powder having a median particle size from about 1 micron to about 100 microns, from about 2 to about 50 microns or even from about 3 microns to about 30 microns, and optionally repeating said layering step;
b.) optionally, treating said particles to remove any materials that would result in said particles having a Relative Jamming Onset of greater than about 14.

In one aspect the particles disclosed herein may be produced by a process comprising:
a.) layering a mass of seeds having:
   (i) a median particle diameter of from about 150 microns to about 1700 microns, from about 200 microns to about 1200 microns, from about 250 microns to about 850 microns or even from about 300 microns to about 600 microns;
   (ii) optionally a size distribution span of from about 1.0 to about 2.0, from about 1.05 to about 1.7, or even from about 1.1 to about 1.5;
   (iii) a seed bulk density of from about 50 grams per liter to about 2000 grams per liter, from about 200 grams per liter to about 1650 grams per liter, from about 350 grams per liter to about 1200 grams per liter or even from about 400 grams per liter to about 850 grams per liter; and
   (iv) optionally, a median particle aspect ratio of from about 1 to about 2, from about 1 to about 1.5, or even from about 1 to about 1.3;
b.) said layering process comprising independently contacting said mass of seeds with a binder having a viscosity of from about 0.5 cp to about 4000 cp, from about 1 cp to about 2000 cp, from about 2 cp to about 1000 cp, from about 5 cp to about 600 cp, or even from about 20 cp to about 400 cp and a layering powder having a median particle size from about 1 micron to about 100 microns, from about 2 to about 50 microns or even from about 3 microns to about 30 microns, and optionally repeating said layering step;
c.) optionally, conducting said process at a layering Stokes Number of from greater than 0 to about 10, from about 0.001 to about 10, or even from about 0.01 to about 5;
d.) optionally, conducting said process at a Coalescence Stokes Number of at least 0.5, from about 1 to about 1000, or even from about 2 to about 1000
e.) optionally treating said particles to remove any materials that would result in said particles having a Relative Jamming Onset of greater than about 14.

In another aspect, the particles disclosed herein may be produced by a process comprising:
a.) layering a mass of seeds with a binder and a layering powder said process comprising independently contacting said mass of seeds with said binder and said layering powder, said processing being conducted at a layering Stokes Number of from greater than 0 to about 10, from about 0.001 to about 10, or even from about 0.01 to about 5; and a Coalescence Stokes Number of at least 0.5, from about 1 to about 1000, or even from about 2 to about 1000; and
b.) optionally layering said mass of seeds one or more times in accordance with the process parameters of a.) above; and
c.) optionally treating said particles to remove any materials that would result in said particles having a Relative Jamming Onset of greater than about 14.

In one aspect, said particles are treated to remove excess binder liquid. In one aspect, said binder is an aqueous solution or dispersion, and the excess binder liquid is water. In one aspect, said treatment includes convective air drying. In one aspect, said convective air drying occurs after the layering process. In one aspect, said layering process is divided into intervals and said convective air drying occurs at the end of each interval. In one aspect, said convective air drying occurs during the layering process. Suitable convective air dryers include fluid beds or fluid bed dryers, available from Niro Inc., Columbia, Md., USA; Kason Corporation, Millburn, N.J., USA; Allgaier Werke GmbH, Uhingen, Germany; Glatt Ingenieurtechnik GmbH, Weimar, Germany; and Bepex International LLC, Minneapolis, Minn., U.S.A. A suitable mixer with integral convective air drying for drying at intervals in the layering process or even drying during layering can be adapted from equipment available from Forberg International AS, Larvik, Norway; and Dynamic Air Inc., St. Paul, Minn., USA by adding one or more layering powder inlets to such equipment.

In one aspect, said independently contacting said mass of seeds with a binder comprising a liquid and a layering powder comprises introducing said binder into a counter-rotating dual-axis paddle mixer having a converging flow zone between the counter-rotating paddle axes such that said binder is directed upward into the converging flow zone between said counter-rotating paddle axes.

In one aspect, said independently contacting said mass of seeds with a binder comprising a liquid and a layering powder comprises introducing said layering powder into a counter-rotating dual-axis paddle mixer having multiple layering powder ingress locations and mixing paddles having a downward trajectory, such that said layering powder is introduced in more than one of said locations in the downward trajectory of the mixing paddles.

In one aspect, the Layering Rate of the process is more than about 5 mass % per minute, more than about 10 mass % per minute, more than about 20 mass % per minute, more than 30 mass % per minute, or even more than about 40 mass % per minute.

In one aspect, the Layering Rate of the process is from about 5 mass % per minute to about 200% per minute.

As it is advantageous to minimize fines and/or over sized products, yet such fines and/or oversized products may still be produced, said particles may be treated to remove fines and oversized products. In one aspect, such fines and oversized product may be removed and then recycled back into the process for further processing. In one aspect, said oversize product may be processed through a cage grinding mill before being recycled back into the process. A suitable grinder for oversize product is available from Stedman Machine Company, Aurora, Ind., USA, Otsuka Iron Works, Ltd., Tokyo, Japan. In one aspect, fines may be removed by screening and/or elutriation of fines, such as attrition products and excess unattached layering powder, in equipment such as, a vibratory screener, fluid bed, airlift, and/or a mixer having supplemental air fluidization. In one aspect, convective air drying with heated air may be incorporated in the air-elutriation step.

In one aspect, fines may be processed through a high-speed grinding mill before being recycled back into the process as layering powder. A suitable high-speed grinding mill is available from Hosokawa Alpine Aktiengesellschaft & Co. OHG, Augsburg, Germany; Netzsch-Feinmahitechnik GmbH, Selb/Bayem, Germany; RSG Incorporated, Sylacauga, Ala., USA.

In one aspect, said particles may be treated by screening out oversized particles using equipment such as a vibratory screener. A vibratory screener suitable for screening out either oversize or undersize particles is available from Sweco, Florence, Kentucky, USA; Kason Corporation, Millburn, N.J., USA; Mogensen GmbH, Wedel/Hamburg, Germany.

In one aspect said layering process of independently contacting said mass of seeds with a binder and a layering powder is selected from the processes of simultaneously contacting a mass of seeds with independent streams of said binder and said layering powder; contacting said mass of seeds in a first location with a stream of said binder and then contacting said seed-binder mixture with a stream of said layering powder in a second location; contacting a mass of seeds with a stream of said layering powder in a first location and then contacting said seed-powder mixture with a stream of said binder in a second location or combination thereof. When more than one layer is required, said contacting process may be repeated one or more times. In one aspect, said layering process may optionally include, but are not limited to, an air-elutriation step to remove any excess fine particles that are not incorporated into layers.

In one aspect, a ploughshare mixer with a chopper located between the ploughs is used where binder ingress is directed just below the chopper location and layering powder ingress is above the chopper location. A suitable ploughshare mixer can be obtained from: Lodige GmbH (Paderborn, Germany); Littleford Day, Inc. (Florence, Ky., U.S.A.). In this aspect, the circumferential convective flow induced by the main ploughshare impeller is such that the seeds are alternately contacted with binder and layering powder. In one aspect, a ploughshare mixer is used where the ingress locations of binder and layering powder are separated in the axial direction. In one aspect, a continuous ploughshare mixer is used with either axial and/or circumferential separations of binder and layering powder.

In one aspect, a counter-rotating dual-axis paddle mixer is used, wherein the counter-rotating shafts are in a horizontal orientation and the paddles attached to the rotating shafts move in an upward trajectory in the space between the parallel counter-rotating shafts and return in a downward trajectory on the outside of the shafts. A suitable counter-rotating dual-axis paddle mixer can be obtained from Forberg International AS, Larvik, Norway; and Dynamic Air Inc., St. Paul, Minn., USA. The motion of the paddles in-between the shafts constitutes a converging flow zone, creating substantial fluidization of the particles in the center of the mixer. During operation of the mixer the tilt of the paddles on each shaft may create opposing convective flow fields in the axial directions generating an additional shear field in the converging flow zone. The downward trajectory of the paddles on the outside of the shafts constitutes a downward convective flow.

In one aspect, the gap between a paddle tip and mixer wall has a narrow clearance below the horizontal plane of the paddle axes, for example a gap clearance of less than about 2 cm. In one aspect, below said horizontal plane, the curvature of the mixer wall contains a volume that is only slightly bigger than the swept volume of the paddles. In one aspect, the narrow gap clearance may be extended above the horizontal axis plane, for example by extending the curvature of the mixer wall or by adding an insert such as a shroud. While not being bound by theory, Applicants believe that said extension of the narrow gap clearance provides a more uniform shear field in the mixer, especially when running at a Froude Number greater than one, i.e., when inertial acceleration of the paddles exceeds gravity. While not being bound by theory, Applicants believe that said extension of the narrow gap clearance above the horizontal axis plane mitigates the potential for build-up of material on the wall, thereby increasing the Product Yield.

In one aspect, a counter-rotating dual-axis paddle mixer is used where binder ingress is via a top-spray in the central fluidized zone and layering powder ingress is at the sides or corners of the mixer into the downward convective flow. In one aspect, a counter-rotating dual-axis paddle mixer is used where the binder ingress is provided such that the binder is added upward into the converging flow zone between the counter-rotating paddle axes, and the layering powder ingress is at a side or corner location such that the layering powder is added in the downward convective flow of the mixer. In one aspect, ingress for binder or layering powder may be provided through an opening in the mixer wall or an opening in a mixer insert such as a shroud. In one aspect, said upward addition of binder into the converging flow zone can be done by adding a binder distributor pipe with one or more holes running parallel to the axial direction of the mixer, where the mixer is modified to allow clearance for said distributor pipe just below the converging flow zone. In one aspect, binder can be added upward into the converging flow zone through one or more binder addition pipes or nozzles, where the mixer is modified to allow clearance of a pipe or nozzle through the mixer wall at a position below the converging flow zone. In one aspect, said layering powder ingress is positioned such that said powder is fed into the downward paddle trajectory of the dual-axis paddle mixer. In these cases, the convective flow induced by the paddle impellers is such that the seeds may be alternately contacted with binder and layering powder in separate locations of the mixer. In one aspect, multiple layering powder ingress locations are provided. While not being bound by theory, Applicants believe that such multiple locations create multiple convective loops in which to alternately contact seeds with binder and layering powder. In addition, while not being bound by theory, Applicants believe that scale-up of the layering process is facilitated by increasing the number of convective loops. While not being bound by theory, Applicants believe that mixer selection may depend on the strength of the seed relative to the shear intensity within the mixer.

In one aspect, said layering step may be repeated a sufficient number of times to increase the particulate mass by a factor of more than about two compared to the initial seed mass, more than about four, or even more than about six times the initial seed mass.

In one aspect, said layering step may be repeated a sufficient number of times to increase particulate mass by a factor of from about 2 to about 100 compared to the initial seed mass.

In one aspect, said layering steps may be conducted in a single mixer batch process.

In one aspect, said layering steps may be conducted in a sequence of two or more batch processes.

In one aspect, said layering steps may be conducted in a sequence of two or more batch process mixers with increasing volumetric capacity to accommodate the increase in product volume.

In one aspect, said layering process may be conducted using a series of one or more mixers. In one aspect, the product granules of a first mixer are used as the seed granules of a following mixer. In one aspect, oversize material may be removed by screening, such oversized material may be reduced in size by milling and such milled material may be transported to, for example by a recycle loop, and used in one or more of the processes mixers as a seed material. In one aspect, said series of mixers is arranged in a continuous process with continuous in-flow of seeds and out-flow of product granules.

In one aspect, said layering process produces acceptable product granules without oversize or undersize tailings. In one aspect, said tailings comprise less than 20 mass % of the processed material, less than 10 mass % or even less than 5% of the processed material. In one aspect, the Product Yield is greater than 80 mass %, greater than 90 mass % or even greater than 95 mass %. In one aspect, the Yield Rate is greater than about 4 mass % per minute, more than about 8 mass % per minute, more than about 16 mass % per minute, more than 24 mass % per minute, more than about 32 mass % per minute, or even more than about 40 mass % per minute.

In one aspect, the mass of seeds and layering powder are introduced into the process at separate times but at substantially identical physical locations.

In one aspect, the process may have an average particle residence time of from about greater than 0 minutes to about 60 minutes, from about 1 minute to about 60 minutes, from about 1 minute to 30 minutes, or even from about 2 minutes to 15 minutes.

In another aspect, Applicants' particles may be made by a process that does not require a mass of seeds. In one aspect, composite particles can be made using an extrusion/spheronization process. Extrusion/spheronization equipment is available from LCI Corporation, Charlotte, N.C., U.S.A. In another aspect, material can be processed from a molten state, atomized, and then congealed into solid particles in a prill-congealing process. In another aspect, a prill-drying process can be used to form particles around a droplet template and then coated with a fine layering powder. Prill-congealing and prill-drying equipment are available from GEA/Niro of Columbia, Md., U.S.A.

As will be appreciated by the skilled artisan, the aforementioned process aspects and those found throughout this specification, including the examples, may be combined in any manner as required to achieve the type and quality of particle that is desired.

Applicants recognized that Stokes numbers can be used to define processing parameters for layering and agglomeration processes. As such, Applicants' processes may be conducted according to the following process parameters: Layering Stokes Number of less than 10, from about 0.001 to about 10 or even from about 0.001 to about 5, and a Coalescence Stokes Number of greater than 0.5, from about 1 to about 1000 or even from about 2 to about 1000. The aforementioned Stokes numbers can be calculated as follows:
Stₘᵢₓₑᵣ=(0.0001)·N·R·ρ·δ/η

The variables in the above equation are specified with units of measurement as follows:
N is the rotational speed of the main agitation impeller shaft in the mixer (revolutions per minute, abbreviated as RPM)
R in radial sweep distance of the main agitation impeller, from the center of the impeller shaft to the tip of the impeller tool, for example a paddle or ploughshare impeller tool (meters, abbreviated as m);
ρ is bulk density of the seed particles (grams/liter, abbreviated as g/l);
η is binder viscosity (centipoises, abbreviated as cp); and
δ is effective particle size used to describe layering or agglomeration (microns, abbreviated as um), where:
   δ_{layering} is defined as 2·(d_{seed}·d_{layering})/(d_{seed}+d_{layering}), and
   δ_{coalescence} is defined as d_{seed}; where
      d_{seed} is the median particle size of the seed material, and
      d_{layering} is the median particle size of the layering powder material.

Based on the above, two sub-forms of the Stokes equation can be defined, one to describe the binding of the layering powder onto the seed particles (St_{layering}), and another to describe the coalescence of seed particles with other seeds (St_{coalescence}).
Layering Stokes Number, St_{layering}=(0.0001)·N·R·ρ·δ_{layering}/η
Coalescence Stokes Number, St_{coalescence}=(0.0001)·N·R·ρ·δ_{coalescence}/η

For the purpose of calculating said Stokes Numbers, the relevant characteristics of seeds, layering powders and binders are based on their measured values before addition to the layering process. In the aspect of a compound layering process conducted in a sequence of two or more mixer stages, Stokes Numbers for each stage are based on the characteristic bulk density and size of the seed material used at the start or entrance or each stage. In the aspect of a layering process using concurrent addition of more than one binder, then the volume-weighted average of the binder viscosity is used for the Stokes Number calculation. In the aspect of a layering process using concurrent addition of more than one layering powder, then the mass-weighted average of the layering powder median particle size is used for the Stokes Number calculation.

Suitable equipment for performing the processes disclosed herein includes paddle mixers, horizontal-axis paddle mixers, dual-axis paddle mixers, counter-rotating dual-axis paddle mixers, ploughshare mixers, ribbon blenders, vertical axis granulators and drum mixers, both in batch and, where available, in continuous process configurations. Such equipment can be obtained from Lodige GmbH (Paderborn, Germany), Littleford Day, Inc. (Florence, Ky., U.S.A.), Dymanic Air (St. Paul, Minn., USA), S. Howes, Inc. (Silver Creek, N.Y., USA), Forberg AS (Larvik, Norway), Glatt Ingenieurtechnik GmbH (Weimar, Germany).

In one aspect, small-scale prototypes are produced using the process. A bench-top vertical-axis mixer may be used to make such prototypes. Suitable equipment for performing the processes disclosed herein includes kitchen mixers, bladed kitchen mixers, food processors, bladed food processors and variable-speed food processors. Such equipment, including Braun, Kenwood, Bosch, Delonghi, Robot Coupe and other commercial brands are available though retail outlets, department stores, appliance stores and restaurant supply stores

Finished Product Comprising Particulates

The finished products of the present invention comprise an embodiment of the particulate disclosed in the present application. While the precise level of particulate that is employed depends on the type and end use of the finished product, in one aspect of Applicants' invention, the finished product may comprise, based on total product weight, a minimum of 50, 60, 70, 80 or even 90 mass percent of the particulates of the present invention—said particulates may comprise one or more distinct particles.

In one aspect, said finished product may have a Relative Jamming Onset of from about 2 to about 14, from about 2.5 to about 12, from about 3 to about 10, or even from about 4 to about 8 particles.

In one aspect, said finished product may have a Relative Jamming Onset of from about 2 to about 14, from about 2.5 to about 12, from about 3 to about 10, or even from about 4 to about 8 particles and a Rapid Stability Relative Jamming Onset of from about 2 to about 18, from about 2 to about 14, from about 2.5 to about 12, from about 3 to about 10, or even from about 4 to about 8 particles. In one aspect, said finished product is: an industrial chemical; an edible food, instant beverage mix, drug or nutriceutical; a pet food and/or pet care product; or a detergent, fabric treatment, personal cleaning, hair care and/or fertilizer product. In one aspect, such finished product may be an automatic dishwashing product.

When the finished product is a cleaning composition, said cleaning compositions disclosed herein are typically formulated such that, during use in aqueous cleaning operations, the wash water will have a pH of between about 6.5 and about 12, or between about 7.5 and 10.5. Particulate dishwashing product formulations that may be used for hand dish washing may be formulated to provide a wash liquor having a pH between about 6.8 and about 9.0. Cleaning products are typically formulated to have a pH of from about 7 to about 12. Techniques for controlling pH at recommended usage levels include, but are not limited to, the use of buffers, alkalis, acids, etc., and are well known to those skilled in the art.

Packaged Product

In one aspect, Applicants' invention may comprise a packaged product comprising a finished product that may comprise an embodiment of the particulates disclosed herein. Said packaged product may contain at least a portion of said particulates and an orifice having a critical gap dimension that is from about greater than the Absolute Jamming Onset of said finished product but less than four times, less than 3 times or less than 2 times said Absolute Jamming Onset. In one aspect, the invention may comprise a packaged product comprising a finished product having a Relative Jamming Onset of greater than 14. In one aspect, the invention may comprise a packaged product comprising a finished product having a Relative Jamming Onset of from about 2 to about 20, from about 2 to about 18, from about 2 to about 16, or even from about 2 to about 15 particles, and a product dosing orifice having a Critical Gap Dimension that is from about 2 mm to about 11 mm, from about 3 mm to about 9 mm, from about 4 mm to about 8 mm or even from about 5 mm to about 7 mm. In one aspect, the packaged product may comprise a container, such as a bottle, bag or carton. In one aspect, at least a portion of said container is transparent. In one aspect, at least 5%, 10%, 20%, 30%, 40%, 50%, 60%, 70%, 80%, 90%, or even 100% of the container's surface area may be transparent. Materials from which said transparent portion may be made include, but are not limited to: polypropylene (PP), polyethylene (PE), polycarbonate (PC), polyamides (PA) and/or polyethylene terephthalate (PETE), polyvinylchloride (PVC); and polystyrene (PS). The transparent portion of said container may have a transmittance of more than 25%, 30%, 40%, 50%, 60% or even more than 70% in the spectrum of 410-800 nm. For purposes of the invention, as long as one wavelength in the visible light range has greater than 25% transmittance, it is considered to be transparent. Thus, the transparent portion of the container may be tinted. The container of the present invention may be of any form or size suitable for storing and packaging cleaning compositions for household use. For example, the container may have any size but usually the container will have a maximal capacity of 0.05 to 15 L, 0.1 to 5 L, 0.2 to 3 L or even 1 to 2 L. Preferably, the container is suitable for easy handling. For example the container may have handle or a part with such dimensions to allow easy lifting or carrying the container with one hand. The container may have a means suitable for pouring material contained in the container and means for reclosing the container. The closing means may be of any form or size but usually will be screwed or clicked on the container to close the container. The closing means may be a cap which can be detached from the container. Alternatively, the cap can still be attached to the container, whether the container is open or closed. The closing means may also be incorporated in the container. In one aspect, said packaged product may be packaged in accordance with the teachings of U.S. published patent application No. 2006/0032872 A1.

Adjunct Detergent Materials

While not essential for the purposes of the present invention, the non-limiting list of adjuncts illustrated hereinafter are suitable for use in the instant compositions and may be desirably incorporated in certain embodiments of the invention, for example to assist or enhance cleaning performance, for treatment of the substrate to be cleaned, or to modify the aesthetics of the cleaning composition as is the case with perfumes, colorants, dyes or the like. The precise nature of these additional components, and levels of incorporation thereof, will depend on the physical form of the composition and the nature of the cleaning operation for which it is to be used. Suitable adjunct materials include, but are not limited to, surfactants, builders, chelating agents, dye transfer inhibiting agents, dispersants, enzymes, and enzyme stabilizers, catalytic materials, bleach activators, hydrogen peroxide, sources of hydrogen peroxide, preformed peracids, polymeric dispersing agents, structurants, clay soil removal/anti-redeposition agents, brighteners, suds suppressors, dyes, fabric hueing agents, perfumes, structure elasticizing agents, fabric softeners, carriers, hydrotropes, processing aids, solvents and/or pigments. In addition to the disclosure below, suitable examples of such other adjuncts and levels of use are found in U.S. Pat. Nos. 5,576,282, 6,306,812 B1 and 6,326,348 B1 that are incorporated by reference.

As stated, the adjunct ingredients are not essential to Applicants' compositions. Thus, certain embodiments of Applicants' compositions do not contain one or more of the following adjuncts materials: surfactants, builders, chelating agents, dye transfer inhibiting agents, dispersants, enzymes, and enzyme stabilizers, catalytic materials, bleach activators, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, processing aids, solvents and/or pigments. However, when one or more adjuncts are present, such one or more adjuncts may be present as detailed below:

Bleaching Agents—The cleaning compositions of the present invention may comprise one or more bleaching agents. Suitable bleaching agents other than bleaching catalysts include, but are not limited to, photobleaches, bleach activators, hydrogen peroxide, sources of hydrogen peroxide, pre-formed peracids and mixtures thereof. In general, when a bleaching agent is used, the compositions of the present invention may comprise from about 0.1% to about 50% or even from about 0.1% to about 25% bleaching agent by weight of the subject cleaning composition. Examples of suitable bleaching agents include, but are not limited to:

(1) preformed peracids: Suitable preformed peracids include, but are not limited to, compounds selected from the group consisting of percarboxylic acids and salts, percarbonic acids and salts, perimidic acids and salts, peroxymonosulfuric acids and salts, for example, Oxone®, and mixtures thereof. Suitable percarboxylic acids include, but are not limited to, hydrophobic and hydrophilic peracids having the formula R-(C=O)O-O-M wherein R is an alkyl group, optionally branched, having, when the peracid is hydrophobic, from 6 to 14 carbon atoms, or from 8 to 12 carbon atoms and, when the peracid is hydrophilic, less than 6 carbon atoms or even less than 4 carbon atoms; and M is a counterion, for example, sodium, potassium or hydrogen;

(2) sources of hydrogen peroxide, for example, inorganic perhydrate salts, including alkali metal salts such as sodium salts of perborate (usually mono- or tetra-hydrate), percarbonate, persulphate, perphosphate, persilicate salts and mixtures thereof. In one aspect of the invention the inorganic perhydrate salts are selected from the group consisting of sodium salts of perborate, percarbonate and mixtures thereof. When employed, inorganic perhydrate salts are typically present in amounts of from 0.05 to 40 wt %, or 1 to 30 wt % of the overall composition and are typically incorporated into such compositions as a crystalline solid that may be coated. Suitable coatings include, but are not limited to, inorganic salts such as alkali metal silicate, carbonate or borate salts or mixtures thereof, or organic materials such as water-soluble or dispersible polymers, waxes, oils or fatty soaps; and

(3) bleach activators having R—(C═O)—L wherein R is an alkyl group, optionally branched, having, when the bleach activator is hydrophobic, from 6 to 14 carbon atoms, or from 8 to 12 carbon atoms and, when the bleach activator is hydrophilic, less than 6 carbon atoms or even less than 4 carbon atoms; and L is leaving group. Examples of suitable leaving groups are benzoic acid and derivatives thereof—especially benzene sulphonate. Suitable bleach activators include, but are not limited to, dodecanoyl oxybenzene sulphonate, decanoyl oxybenzene sulphonate, decanoyl oxybenzoic acid or salts thereof, 3,5,5-trimethyl hexanoyloxybenzene sulphonate, tetraacetyl ethylene di amine (TAED) and nonanoyloxybenzene sulphonate (NOBS). Suitable bleach activators are also disclosed in WO 98/17767. While any suitable bleach activator may be employed, in one aspect of the invention the subject cleaning composition may comprise NOBS, TAED or mixtures thereof.

When present, the peracid and/or bleach activator is generally present in the composition in an amount of from about 0.1 to about 60 wt %, from about 0.5 to about 40 wt % or even from about 0.6 to about 10 wt % based on the composition. One or more hydrophobic peracids or precursors thereof may be used in combination with one or more hydrophilic peracid or precursor thereof.

The amounts of hydrogen peroxide source and peracid or bleach activator may be selected such that the molar ratio of available oxygen (from the peroxide source) to peracid is from 1:1 to 35:1, or even 2:1 to 10:1.

Surfactants—The cleaning compositions according to the present invention may comprise a surfactant or surfactant system wherein the surfactant can be selected from nonionic surfactants, anionic surfactants, cationic surfactants, ampholytic surfactants, zwitterionic surfactants, semi-polar nonionic surfactants and mixtures thereof. When present, surfactant is typically present at a level of from about 0.1% to about 60%, from about 1% to about 50% or even from about 5% to about 40% by weight of the subject composition.

Builders—The cleaning compositions of the present invention may comprise one or more detergent builders or builder systems. When a builder is used, the subject composition will typically comprise at least about 1%, from about 5% to about 60% or even from about 10% to about 40% builder by weight of the subject composition.

Builders include, but are not limited to, the alkali metal, ammonium and alkanolammonium salts of polyphosphates, alkali metal silicates, alkaline earth and alkali metal carbonates, aluminosilicate builders and polycarboxylate compounds, ether hydroxypolycarboxylates, copolymers of maleic anhydride with ethylene or vinyl methyl ether, 1,3,5-trihydroxy benzene-2,4,6-trisulphonic acid, and carboxymethyloxysuccinic acid, the various alkali metal, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof.

Chelating Agents—The cleaning compositions herein may contain a chelating agent. Suitable chelating agents include, but are not limited to, copper, iron and/or manganese chelating agents and mixtures thereof. When a chelating agent is used, the subject composition may comprise from about 0.005% to about 15% or even from about 3.0% to about 10% chelating agent by weight of the subject composition.

Dye Transfer Inhibiting Agents—The cleaning compositions of the present invention may also include, but are not limited to, one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Brighteners—The cleaning compositions of the present invention can also contain additional components that may tint articles being cleaned, such as fluorescent brighteners. Suitable fluorescent brightener levels include lower levels of from about 0.01, from about 0.05, from about 0.1 or even from about 0.2 wt % to upper levels of 0.5 or even 0.75 wt %.

Dispersants—The compositions of the present invention can also contain dispersants. Suitable water-soluble organic materials include, but are not limited to, the homo- or co-polymeric acids or their salts, in which the polycarboxylic acid comprises at least two carboxyl radicals separated from each other by not more than two carbon atoms.

Enzymes—The cleaning compositions can comprise one or more enzymes which provide cleaning performance and/or fabric care benefits. Examples of suitable enzymes include, but are not limited to, hemicellulases, peroxidases, proteases, cellulases, xylanases, lipases, phospholipases, esterases, cutinases, pectinases, mannanases, pectate lyases, keratinases, reductases, oxidases, phenoloxidases, lipoxygenases, ligninases, pullulanases, tannases, pentosanases, malanases, β-glucanases, arabinosidases, hyaluronidase, chondroitinase, laccase, and amylases, or mixtures thereof. A typical combination is an enzyme cocktail that may comprise, for example, a protease and lipase in conjunction with amylase. When present in a cleaning composition, the aforementioned enzymes may be present at levels from about 0.00001% to about 2%, from about 0.0001% to about 1% or even from about 0.001% to about 0.5% enzyme protein by weight of the composition.

Enzyme Stabilizers—Enzymes for use in detergents can be stabilized by various techniques. The enzymes employed herein can be stabilized by the presence of water-soluble sources of calcium and/or magnesium ions in the finished compositions that provide such ions to the enzymes. In case of aqueous compositions comprising protease, a reversible protease inhibitor, such as a boron compound, can be added to further improve stability.

Catalytic Metal Complexes—Applicants' cleaning compositions may include catalytic metal complexes. One type of metal-containing bleach catalyst is a catalyst system comprising a transition metal cation of defined bleach catalytic activity, such as copper, iron, titanium, ruthenium, tungsten, molybdenum, or manganese cations, an auxiliary metal cation having little or no bleach catalytic activity, such as zinc or aluminum cations, and a sequestrate having defined stability constants for the catalytic and auxiliary metal cations, particularly ethylenediaminetetraacetic acid, ethylenediaminetetra(methylenephosphonic acid) and water-soluble salts thereof. Such catalysts are disclosed in U.S. Pat. No. 4,430,243.

If desired, the compositions herein can be catalyzed by means of a manganese compound. Such compounds and levels of use are well known in the art and include, but are not limited to, for example, the manganese-based catalysts disclosed in U.S. Pat. No. 5,576,282.

Cobalt bleach catalysts useful herein are known, and are described, for example, in U.S. Pat. No. 5,597,936; U.S. Pat. No. 5,595,967. Such cobalt catalysts are readily prepared by known procedures, such as taught for example in U.S. Pat. No. 5,597,936, and U.S. Pat. No. 5,595,967.

Compositions herein may also suitably include a transition metal complex of ligands such as bispidones (WO 05/042532 A1) and/or macropolycyclic rigid ligands—abbreviated as “MRLs”. As a practical matter, and not by way of limitation, the compositions and processes herein can be adjusted to provide on the order of at least one part per hundred million of the active MRL species in the aqueous washing medium, and will typically provide from about 0.005 ppm to about 25 ppm, from about 0.05 ppm to about 10 ppm, or even from about 0.1 ppm to about 5 ppm, of the MRL in the wash liquor.

Suitable transition-metals in the instant transition-metal bleach catalyst include, but are not limited to, for example, manganese, iron and chromium. Suitable MRLs include, but are not limited to, 5,12-diethyl-1,5,8,12-tetraazabicyclo[6.6.2]hexadecane.

Suitable transition metal MRLs are readily prepared by known procedures, such as taught for example in WO 00/32601, and U.S. Pat. No. 6,225,464.

Processes of Making Compositions

The compositions of the present invention can be formulated into any suitable form and prepared by any process chosen by the formulator, non-limiting examples of which are described in Applicants' examples and in U.S. Pat. No. 4,990,280; U.S. 20030087791A1; U.S. 20030087790A1; U.S. 20050003983A1; U.S. 20040048764A1; U.S. Pat. No. 4,762,636; U.S. Pat. No. 6,291,412; U.S. 20050227891A1; EP 1070115A2; U.S. Pat. No. 5,879,584; U.S. Pat. No. 5,691,297; U.S. Pat. No. 5,574,005; U.S. Pat. No. 5,569,645; U.S. Pat. No. 5,565,422; U.S. Pat. No. 5,516,448; U.S. Pat. No. 5,489,392; U.S. Pat. No. 5,486,303 all of which are incorporated herein by reference.

Method of Using Cleaning Compositions

The present invention includes a method for cleaning and /or treating a situs inter alia a surface or fabric. Such method includes the steps of contacting an embodiment of Applicants' cleaning composition, in neat form or diluted in a wash liquor, with at least a portion of a surface or fabric then optionally rinsing such surface or fabric. The surface or fabric may be subjected to a washing step prior to the aforementioned rinsing step. For purposes of the present invention, washing includes but is not limited to, scrubbing, and mechanical agitation. As will be appreciated by one skilled in the art, the cleaning compositions of the present invention are ideally suited for use in laundry applications. Accordingly, the present invention includes a method for laundering a fabric. The method may comprise the steps of contacting a fabric to be laundered with a said cleaning laundry solution comprising at least one embodiment of Applicants' cleaning composition, cleaning additive or mixture thereof. The fabric may comprise most any fabric capable of being laundered in normal consumer use conditions. The solution preferably has a pH of from about 8 to about 10.5. The compositions may be employed at concentrations of from about 500 ppm to about 15,000 ppm in solution. The water temperatures typically range from about 5° C. to about 90° C. The water to fabric ratio is typically from about 1:1 to about 30:1.

### Test Methods

It is understood that the test methods that are disclosed in the Test Methods Section of the present application must be used to determine the respective values of the parameters of Applicants' inventions as such inventions are described and claimed herein.

1.) Layering Powder Median Particle Size Test

This test method must be used to determine a layering powder's median particle size. The layering powder's particle size test is determined in accordance with ISO 8130-13, “Coating powders—Part 13: Particle size analysis by laser diffraction.” A suitable laser diffraction particle size analyzer with a dry-powder feeder can be obtained from Horiba Instruments Incorporated of Irvine, Calif., U.S.A.; Malvern Instruments Ltd of Worcestershire, UK; Sympatec GmbH of Clausthal-Zellerfeld, Germany; and Beckman-Coulter Incorporated of Fullerton, Calif., U.S.A.

The results are expressed in accordance with ISO 9276-1:1998, “Representation of results of particle size analysis—Part 1: Graphical Representation”, Figure A.4, “Cumulative distribution Q₃ plotted on graph paper with a logarithmic abscissa.” The median particle size is defined as the abscissa value at the point where the cumulative distribution (Q3) is equal to 50 percent.

2.) Binder Component Viscosity Test

This test method must be used to determine binder component viscosity.

The binder component viscosity is determined using an apparent viscosity obtained by the Brookfield test method. A suitable viscometer, for example Brookfield type LV (LVT or LVDV series) with UL adapter, can be obtained from Brookfield Engineering Laboratories, Inc., Middleboro, Mass., USA. The binder component viscosity test is conducted in accordance with the Brookfield Operating Manual, following the guidelines of ISO 2555, second edition published Feb. 1, 1989 and reprinted with corrections Feb. 1, 1990, “Plastics—resins in the liquid state or as emulsions or dispersions—Determination of apparent viscosity by the Brookfield Test method,” with the following qualifications:
a.) A Brookfield LV series viscometer with UL adapter is used.
b.) A rotational frequency of 60 revolutions per minute is used. The spindle is chosen in accordance with the permitted operating range specified in Clause 4 of ISO 2555. In the case where the rotational frequency of 60 revolutions per minute cannot be used based on the permitted operating range, then the highest speed that is less than 60 revolutions per minute and is in accordance with the permitted range of Clause 4 shall be used.
c.) The viscosity measurement is performed at the same binder component temperature at which the binder component is introduced into the layering process.
   3.) Seed Material Median Particle Size and Distribution Span Test

This test method must be used to determine seed material median particle size.

The seed material particle size test is conducted to determine the median particle size of the seed material using ASTM D 502-89, “Standard Test Method for Particle Size of Soaps and Other Detergents”, approved May 26, 1989, with a further specification for sieve sizes used in the analysis. Following section 7, “Procedure using machine-sieving method,” a nest of clean dry sieves containing U.S. Standard (ASTM E 11) sieves #8 (2360 um), #12 (1700 um), #16 (1180 um), #20 (850 um), #30 (600 um), #40 (425 um), #50 (300 um), #70 (212 um), #100 (150 um) is required. The prescribed Machine-Sieving Method is used with the above sieve nest. The seed material is used as the sample. A suitable sieve-shaking machine can be obtained from W.S. Tyler Company of Mentor, Ohio, U.S.A.

The data are plotted on a semi-log plot with the micron size opening of each sieve plotted against the logarithmic abscissa and the cumulative mass percent (Q₃) plotted against the linear ordinate. An example of the above data representation is given in ISO 9276-1:1998, “Representation of results of particle size analysis—Part 1: Graphical Representation”, Figure A.4. The seed material median particle size (D₅₀), for the purpose of this invention, is defined as the abscissa value at the point where the cumulative mass percent is equal to 50 percent, and is calculated by a straight line interpolation between the data points directly above (a50) and below (b50) the 50% value using the following equation:
D₅₀=10ˆ[Log(Dₐ₅₀)−(Log(Dₐ₅₀)−Log(D_{b50}))*(Qₐ₅₀−50%)/(Qₐ₅₀−Q_{b50})]
where Qₐ₅₀ and Q_{b50} are the cumulative mass percentile values of the data immediately above and below the 50^{th} percentile, respectively; and Dₐ₅₀ and D_{b50} are the micron sieve size values corresponding to these data.

In the event that the 50^{th} percentile value falls below the finest sieve size (150 um) or above the coarsest sieve size (2360 um), then additional sieves must be added to the nest following a geometric progression of not greater than 1.5, until the median falls between two measured sieve sizes.

The Distribution Span of the Seed Material is a measure of the breadth of the seed size distribution about the median. It is calculated according to the following:
Span=(D₈₄/D₅₀+D₅₀/D₁₆)/2
Where D₅₀ is the median particle size and D₈₄ and D₁₆ are the particle sizes at the sixteenth and eighty-fourth percentiles on the cumulative mass percent retained plot, respectively.
In the event that the D₁₆ value falls below the finest sieve size (150 um), then the span is calculated according to the following:
   Span=(D₈₄/D₅₀).
In the event that the D₈₄ value falls above the coarsest sieve size (2360 um), then the span is calculated according to the following:
   Span=(D₅₀/D₁₆).
In the event that the D₁₆ value falls below the finest sieve size (150 um) and the D₈₄ value falls above the coarsest sieve size (2360 um), then the distribution span is taken to be a maximum value of 5.7.
   4.) Bulk Density Test

The seed material bulk density is determined in accordance with Test Method B, Loose-fill Density of Granular Materials, contained in ASTM Standard E727-02, “Standard Test Methods for Determining Bulk Density of Granular Carriers and Granular Pesticides,” approved Oct. 10, 2002.

5.) Flowable Particle Mass Based Cumulative Particle Size Distribution Test

This test method must be used to determine the median particle size (D₅₀) and the 30^{th} percentile particle size (D₃₀) of the flowable particulate. This test follows the same procedure that is specified for the “Seed Material Median Particle Size Test” described above except that the method is used to measure:
a) Selected particle size percentiles of the flowable particulate, and
b) Selected particle size percentiles of the full admixed composition containing the flowable particulate.

In part (a), the “Seed Material Median Particle Size Test” is performed using the flowable particle as the sample instead of the seed material. The median particle size (D₅₀) is calculated in the same manner. In addition, the 30^{th} percentile particle size (D₃₀) is defined as the abscissa value at the point where the cumulative mass percent is equal to 30 percent, and is calculated by a straight line interpolation between the data points directly above (a30) and below (b30) the 30% value using the following equation:
D₃₀=10ˆ[Log(Dₐ₃₀)−(Log(Dₐ₃₀)−Log(D_{b30}))*(Qa₃₀−30%)/(Qₐ₃₀−Q_{b30})]
where Qₐ₃₀ and Q_{b30} are the cumulative mass percentile values of the data immediately above and below the 30^{th} percentile, respectively; and Dₐ₃₀ and D_{b30} are the micron sieve size values corresponding to these data.

In the event that the 30^{th} percentile value falls below the finest sieve size (150 um), then additional sieves must be added to the nest following a geometric progression of not greater than 1.5, until the 30^{th} percentile falls between two measured sieve sizes.

In part (b), the procedure of part (a) above is used with the full admixed composition instead of the flowable particulate.

6.) Jamming Onset Test

Jamming Onsets are measured using a Flodex™ instrument supplied by Hanson Research Corporation, Chatsworth, Calif., USA. As used in this test method the term “Hopper” refers to the Cylinder Assembly of the Flodex™ instrument; the term “orifice” refers to the hole in the center of the Flow Disk that is used in a flow test; the symbol “B” refers to the diameter of the orifice in the Flow Disk used in the test; and the symbol “b” refers to the dimensionless orifice size, as defined by the ratio of the orifice diameter to the 30^{th} percentile particle size (D₃₀) specified in Applicant's Test Method #5 titled “Flowable Particle Mass Based Cumulative Particle Size Distribution Test”, b=B/D₃₀.

The Flodex™ instrument is operated in accordance with the instructions contained in the Flodex™ operation manual version 21-101-000 rev. C 2004-03 with the following exceptions:
a.) The suitable container that is used to collect the material that is tested is tared on a balance with 0.01 gram precision before the start of the test, and used subsequently to measure the mass of particulate discharge from the Hopper in step c, below.
b.) Sample preparation. A bulk sample of particles is suitably riffled to provide a sub-sample of 150 ml loose-fill volume. The appropriate sample mass can be determined by measuring the loose fill density specified in Test Method #4, titled “Bulk Density Test”, and then multiplying by the target volume (150 ml). The mass of the sample (sample mass) is recorded before the start of each test measurement. As the test is non destructive, the same sample may be used repeatedly. The entire sample must be discharged, e.g., by inverting the hopper, and then re-loaded before each measurement.
c.) Starting with the smallest orifice size (typically 4 mm unless a smaller orifice is necessary), three repeat measurements are taken for each orifice size. For each measurement, the sample is loaded into the Hopper and allowed to rest for a rest interval of about 30 seconds before the orifice is opened according to the procedure described in the Flodex™ Operation Manual. The sample is allowed to discharge into the tared container for a period of at least 60 seconds. After this 60 second period and once the flow stops and remains stopped for 30 seconds (i.e., no more than 0.1 mass % of the material is discharged over the 30 second stop interval), then the mass of discharged material is measured, the orifice is closed and the Hopper is fully emptied by inverting the Hopper assembly or removing the flow disk. Note: if the flow stops and then re-starts during the 30 second stop interval, then the stop interval clock must be re-started at zero at the next flow stoppage. For each measurement, the mass % discharged is calculated according to the formula: (mass % discharged)=100*(mass discharged)/(sample mass). The average of the three mass % discharged measurements is plotted as a function of the dimensionless orifice size (b=B/D₃₀), with the mass % discharged on the ordinate and the dimensionless orifice size on the abscissa. This procedure is repeated using incrementally larger orifice sizes until the hopper discharges without jamming for three consecutive times, as per the description of a “positive result” in the Flodex™ Operation Manual.
d.) The plotted data are then linearly interpolated to find the Relative Jamming Onset (Jᵣₑₗ), which is defined as the value of the dimensionless orifice size at the point of 25 mass % average discharge. This is determined by the abscissa value (b) at the point where the interpolation is equal to 25 mass % discharge. If the average mass % discharge exceeds 25% for the starting orifice, then flow disks with smaller orifices must be obtained and the test repeated starting with the smaller orifice. Flow disks with smaller orifices such as 3.5, 3.0, 2.5 or even 2.0 mm can be obtained as custom parts from Hanson Research Corporation.
e) The Absolute Jamming Onset (J_{abs}) is defined as the product of the Relative Jamming Onset and the D₃₀ particle size, J_{abs}=Jᵣₑₗ* D₃₀.
   7.) Rapid Stability Relative Jamming Onset Test

The Rapid Stability Relative Jamming Onset Test is a measure of the physical stability of the particulate flow property on exposure to a warm, humid environment. The test is performed in accordance with Test Method #6 titled “Jamming Onset Test” with the following qualifications:
a) An environmental aging step is added, whereby the 150 ml sample of Test Method #6 is placed in a 250 ml beaker and then aged by placing the uncovered sample in an environmental test chamber at 27 degrees Celsius and 60% relative humidity for a period of 48 hours. The 250 ml beaker is straight sided with an open top and an inside diameter of about 6.5 cm. A suitable constant temperature and humidity chamber may be obtained from Lunaire Environmental Products, New Columbia, Pa., USA; Weiss-Gallenkamp, Loughborough, UK, ESPEC, Hudsonville, Mich., USA.
b) The remainder of the Jamming Onset Test is performed on the aged sample. After removing an aged sample from the environmental chamber, it may be used in the Jamming Onset Test for a time period not to exceed 20 minutes. If additional time is required to complete the test, then multiple aged samples must be prepared. Note it may be necessary to tap the beaker or even break up the aged particulate sample using a spatula in order to discharge it from the beaker at the end of the aging period.
c) The Rapid Stability Relative Jamming Onset is obtained according to the Relative Jamming Onset calculation, using the D₃₀ value of the particulate measured before aging in the 48 hour environmental test.
   8.) Particle Aspect Ratio Test

The particle aspect ratio is defined as the ratio of the particle's major axis diameter (dₘₐⱼₒᵣ) relative to the particle's minor axis diameter (dₘᵢₙₒᵣ), where the major and minor axis diameters are the long and short sides of a rectangle that circumscribes a 2-dimensional image of the particle at the point of rotation where the short side of the rectangle is minimized. The 2-dimensional image is obtained using a suitable microscopy technique. For the purpose of this method, the particle area is defined to be the area of the 2-dimensional particle image. In order to determine the aspect ratio distribution and the median particle aspect ratio, a suitable number of representative 2-dimensional particle images must be acquired and analyzed. For the purpose of this test, a minimum of 5000 particle images is required. In order to facilitate collection and image analysis of this number of particles, an automated imaging and analysis system is recommended. Such systems can be obtained from Malvern Instruments Ltd., Malvern, Worcestershire, United Kingdom; Beckman Coulter, Inc., Fullerton, Calif., USA; JM Canty, Inc., Buffalo, N.Y., USA; Retsch Technology GmbH, Haan, Germany; and Sympatec GmbH, Clausthal-Zellerfeld, Germany.

A suitable sample of particles is obtained by riffling. The sample is then processed and analyzed by the image analysis system, to provide a list of particles containing major and minor axis attributes. The aspect ratio (AR) of each particle is calculated according to the ratio of the particle's major and minor axis,
AR=dₘₐⱼₒᵣ/dₘᵢₙₒᵣ.

The list of data are then sorted in ascending order of particle aspect ratio and the cumulative particle area is calculated as the running sum of particle areas in the sorted list. The particle aspect ratio is plotted against the abscissa and the cumulative particle area against the ordinate. The median particle aspect ratio is the abscissa value at the point where the cumulative particle area is equal to 50% of the total particle area of the distribution.

### EXAMPLES

### Example 1

### Seed Materials

Seed materials are commonly available as granular grades of feedstock materials with a particle size, size distribution, aspect ratio and density that is within the description of the invention. Suitable single-component seeds include granular grades of sodium tripolyphosphate, sodium sulfate, sodium carbonate, sodium silicate, monocalcium phosphate, dicalcium phosphate, sodium bisulfate, sodium citrate, citric acid, lactose, sugar, whey, and starch granules. Such seeds may be useful for a broad range of applications.

Examples of composite compositions for use as detergent seeds are given in Tables 1A and 1B, Intermediate Detergent Compositions, Columns a through x. Such composite seeds are prepared by an independent detergent granulation process such as mechanical agglomeration, spray drying or extrusion, and then classified to meet the seed size specification. Such processes for making intermediate granular compositions are well known to those familiar with the art.

In one aspect, an intermediate detergent composition (e.g., as per Table 1) may be classified into two portions, one portion that is suitable for use as seeds, and a second portion that is not needed or not suitable for use as seeds. The second portion may then be milled into a fine powder that is suitable for layering. In this way, the total amount of the intermediate composition can be consumed in the layering process, either as seeds or as layering powder. Further, the portioning of an intermediate material into seed and layering fractions provides for control of the layering process, relative to the ratio of the binder and layering powder, as well as control over the product attributes, for example the layered particle size relative to the initial seed size.

TABLE 1A
Intermediate detergent compositions (by mass)

| Ingredient* | (a) | (b) | (c) | (d) | (e) | (f) | (g) | (h) | (i) | (j) | (k) | (l) |
|---|---|---|---|---|---|---|---|---|---|---|---|---|
| 1 | 16.7 | 10.5 | 13.6 | 0.0 | 0.0 | 0.0 | 0.0 | 0.0 | 0.0 | 0.0 | 0.0 | 0.0 |
| 2 | 0.0 | 0.0 | 0.0 | 0.0 | 0.0 | 35.0 | 0.0 | 20.0 | 0.0 | 40.8 | 0.0 | 0.0 |
| 3 | 31.3 | 27.9 | 40.3 | 46.5 | 16.5 | 30.0 | 37.0 | 45.0 | 46.8 | 23.5 | 36.5 | 34.2 |
| 4 | 35.8 | 47.9 | 33.0 | 0.0 | 54.5 | 0.0 | 37.0 | 0.0 | 0.0 | 0.0 | 27.7 | 20.0 |
| 5 | 12.8 | 8.8 | 10.6 | 0.0 | 1.5 | 0.0 | 0.0 | 0.0 | 8.2 | 0.0 | 0.0 | 0.0 |
| 6 | 0.0 | 0.0 | 0.0 | 8.0 | 15.0 | 0.0 | 19.0 | 0.0 | 0.0 | 0.0 | 19.9 | 19.8 |
| 7 | 0.0 | 0.0 | 0.0 | 0.0 | 0.0 | 20.0 | 0.0 | 20.0 | 0.0 | 23.5 | 0.0 | 0.0 |
| 8 | 0.0 | 0.0 | 0.0 | 4.0 | 0.0 | 0.0 | 0.0 | 0.0 | 15.0 | 0.0 | 0.0 | 0.0 |
| 9 | 0.0 | 2.0 | 1.0 | 36.0 | 0.0 | 4.0 | 0.0 | 7.5 | 27.5 | 0.0 | 10.2 | 0.0 |
| 10 | 0.0 | 0.0 | 0.0 | 0.0 | 10.5 | 0.0 | 4.5 | 0.0 | 0.0 | 0.0 | 0.0 | 17.1 |
| 11 | 0.0 | 0.0 | 0.0 | 0.0 | 0.0 | 1.0 | 0.0 | 1.0 | 0.0 | 1.8 | 2.1 | 0.0 |
| 12 | 0.8 | 1.0 | 0.4 | 0.0 | 0.0 | 0.0 | 0.0 | 0.0 | 0.0 | 0.0 | 0.0 | 0.0 |
| 13 | 0.0 | 0.0 | 0.0 | 0.0 | 0.5 | 0.2 | 0.5 | 0.5 | 0.0 | 0.0 | 0.8 | 0.9 |
| 14 | 0.0 | 0.0 | 0.0 | 0.0 | 0.0 | 0.0 | 0.0 | 0.0 | 0.0 | 0.0 | 0.0 | 2.9 |
| 15 | 2.6 | 1.9 | 1.1 | 5.5 | 1.5 | 9.8 | 2.0 | 6.0 | 2.5 | 10.4 | 2.8 | 5.1 |

*Table 1A ingredient list: 1) sodium tripolyphosphate; 2) sodium aluminosilicate, zeolite structure; 3) sodium carbonate; 4) sodium sulfate; 5) sodium silicate; 6) sodium alkyl benzene sulfonate; 7) sodium alkyl sulfate; 8) sodium alkyl ethoxysulfate; 9) sodium polyacrylate polymer; 10) sodium acrylic-maleic copolymer; 11) polyethylene glycol 4000; 12) linear alcohol alkoxylate; 13) optical brightener; 14) carboxymethyl cellulose; 15) moisture and raw material by products.

TABLE 1B
Intermediate detergent compositions (by mass)

| Ingredient* | (m) | (n) | (o) | (p) | (q) | (r) | (s) | (t) | (u) | (v) | (w) | (x) |
|---|---|---|---|---|---|---|---|---|---|---|---|---|
| 1 | 0.0 | 0.0 | 0.0 | 0.0 | 0.0 | 0.0 | 0.0 | 0.0 | 0.0 | 0.0 | 0.0 | 0.0 |
| 2 | 0.1 | 0.0 | 0.0 | 0.0 | 0.0 | 0.0 | 0.0 | 0.0 | 0.0 | 0.0 | 0.0 | 0.0 |
| 3 | 71.2 | 57.0 | 36.3 | 62.0 | 24.3 | 32.4 | 28.2 | 18.5 | 42.1 | 28.3 | 8.4 | 15.7 |
| 4 | 0.0 | 18.0 | 27.4 | 0.0 | 51.4 | 28.1 | 22.7 | 37.0 | 23.4 | 44.8 | 56.2 | 69.4 |
| 5 | 0.0 | 0.0 | 5.1 | 0.0 | 3.4 | 5.0 | 4.8 | 4.0 | 8.1 | 3.8 | 5.4 | 2.1 |
| 6 | 18.4 | 25.0 | 0.0 | 0.0 | 0.0 | 0.0 | 0.0 | 0.0 | 0.0 | 0.0 | 0.0 | 0.0 |
| 7 | 0.0 | 0.0 | 0.0 | 0.0 | 0.0 | 1.2 | 0.0 | 0.8 | 1.1 | 0.0 | 0.0 | 0.0 |
| 8 | 6.9 | 0.0 | 5.5 | 36.0 | 3.7 | 4.3 | 6.1 | 2.3 | 3.3 | 7.7 | 0.4 | 4.3 |
| 9 | 0.7 | 0.0 | 0.0 | 0.0 | 0.0 | 0.0 | 0.0 | 0.0 | 0.0 | 0.0 | 0.0 | 0.0 |
| 10 | 0.0 | 0.0 | 0.0 | 0.0 | 0.0 | 0.0 | 0.0 | 0.0 | 0.0 | 0.0 | 0.0 | 0.0 |
| 11 | 0.0 | 0.0 | 3.6 | 0.0 | 2.4 | 2.7 | 4.1 | 3.5 | 4.9 | 6.0 | 1.6 | 3.3 |
| 12 | 0.0 | 0.0 | 0.0 | 0.0 | 0.0 | 17.3 | 20.2 | 24.7 | 0.0 | 0.0 | 21.8 | 0.0 |
| 13 | 0.0 | 0.0 | 9.1 | 0.0 | 6.1 | 1.0 | 0.0 | 0.0 | 0.0 | 0.0 | 0.0 | 0.0 |
| 14 | 0.0 | 0.0 | 0.0 | 0.0 | 0.0 | 2.1 | 7.1 | 0.0 | 8.6 | 0.0 | 0.0 | 0.0 |
| 15 | 0.0 | 0.0 | 0.3 | 0.0 | 0.2 | 0.4 | 0.3 | 0.4 | 0.5 | 0.4 | 0.0 | 0.2 |
| 16 | 2.7 | 0.0 | 12.7 | 2.0 | 8.5 | 5.5 | 6.5 | 8.8 | 8.0 | 9.0 | 6.2 | 5.0 |

*Table 1B ingredient list: 1) sodium tripolyphosphate; 2) sodium aluminosilicate, zeolite structure; 3) sodium carbonate; 4) sodium sulfate; 5) sodium silicate; 6) sodium alkyl ethoxysulfate; 7) sodium polyacrylate polymer; 8) sodium acrylic-maleic copolymer; 9) linear alcohol alkoxylate; 10) carboxymethyl cellulose; 11) nonionic surfactant; 12) sodium citrate; 13) MGDA; 14) GLDA; 15) HEDP; 16) moisture and raw material by products.

### Example 2

### Layering Powders

While suitable layering powders may be available directly as powder-grade raw materials, supplemental comminution may be necessary to reduce the particle size to the desired size range as per the description of the invention, for example, using a high speed pin mill.

The composition of the layering powder depends on the product application. Layering powders may provide physical and/or chemical adsorption of the liquid binder within the layer structure. When using reactive or aqueous binders, it is preferred that at least one component of the layering powder include a material that is capable of reacting with the binder; in doing so, converting the binder to a solid or semi-solid phase. For example, the layering powder may participate in an acid-base or hydration reaction with other materials or intermediates in the layering process. For example, when using an aqueous binder, it is desired that the layering powder include at least one hydratable material.

Examples of suitable layering materials include, but are not limited to, materials selected from the group consisting of sugars, acetates, citrates, sulfates, carbonates, borates, phosphates, acidic precursors and mixtures thereof. Examples of sugars and carbohydrate salts include, but are not limited to, lactose, calcium lactate, and trehalose. Examples of acetates include, but are not limited to, magnesium acetate, Mg(CH₃COO)₂; and sodium acetate, NaCH₃COO. Examples of citrates include, but are not limited to, sodium citrate, C₆H₅O₇Na₃; and citric acid, C₆H₈O₇. Examples of sulfates include, but are not limited to, magnesium sulfate, MgSO₄; and sodium sulfate, Na₂SO₄. Examples of carbonates include, but are not limited to, sodium carbonate, Na₂CO₃; potassium carbonate, K₂CO₃. Examples of borates include, but are not limited to, sodium borate, Na₂B₄O₇. Examples of phosphates include, but are not limited to, sodium phosphate dibasic, Na₂HPO₄; and sodium tripolyphosphate, Na₅P₃O₁₀. Layering powders containing such materials may be introduced to the layering process as substantially anhydrous salts. While not being bound by theory, it is believed that their conversion to stable hydrate phases provides a mechanism for the removal of binder moisture and enables processing to proceed with improved control. If the hydration capacity of the material is sufficient, the process can be done without the requirement of a drying step.

Additional active layering powder materials for detergent applications include, but are not limited to, materials selected from the group consisting of surfactants, soluble polymers, builders, buffering agents, optical brighteners and mixtures thereof. In one aspect, the layering powder is made by milling an intermediate detergent composition, for example, the compositions as given in Tables 1A and 1B to produce compositions in rows 7-15 of Table 2A and rows 12-16 of Table 2B.

TABLE 2A
Layering Powder Compositions (by mass)

| Ingredients* | (a) | (b) | (c) | (d) | (e) | (f) | (g) | (h) | (i) | (j) | (k) | (l) |
|---|---|---|---|---|---|---|---|---|---|---|---|---|
| 1 | 54.4 | 0.0 | 0.0 | 27.0 | 0.0 | 0.0 | 0.0 | 0.0 | 0.0 | 0.0 | 0.0 | 0.0 |
| 2 | 0.0 | 18.9 | 0.0 | 0.0 | 7.8 | 8.5 | 0.0 | 25.6 | 2.8 | 10.4 | 3.4 | 0.0 |
| 3 | 45.2 | 47.2 | 11.1 | 21.3 | 49.8 | 54.0 | 57.0 | 45.5 | 28.4 | 41.6 | 42.7 | 0.0 |
| 4 | 0.0 | 0.0 | 33.3 | 0.0 | 0.0 | 0.0 | 0.0 | 0.0 | 0.0 | 0.0 | 0.0 | 0.0 |
| 5 | 0.0 | 0.0 | 0.0 | 0.0 | 0.0 | 0.0 | 0.0 | 0.0 | 0.0 | 0.0 | 0.0 | 18.5 |
| 6 | 0.0 | 0.0 | 0.0 | 0.0 | 0.0 | 0.0 | 0.0 | 0.0 | 0.0 | 0.0 | 0.0 | 2.0 |
| 7 | 0.0 | 0.0 | 0.0 | 51.2 | 0.0 | 0.0 | 0.0 | 0.0 | 0.0 | 0.0 | 0.0 | 0.0 |
| 8 | 0.0 | 0.0 | 0.0 | 0.0 | 0.0 | 0.0 | 42.6 | 0.0 | 0.0 | 0.0 | 0.0 | 0.0 |
| 9 | 0.0 | 0.0 | 0.0 | 0.0 | 40.7 | 35.8 | 0.0 | 0.0 | 0.0 | 0.0 | 0.0 | 0.0 |
| 10 | 0.0 | 0.0 | 0.0 | 0.0 | 0.0 | 0.0 | 0.0 | 0.0 | 68.2 | 0.0 | 0.0 | 0.0 |
| 11 | 0.0 | 0.0 | 0.0 | 0.0 | 0.0 | 0.0 | 0.0 | 23.5 | 0.0 | 0.0 | 0.0 | 0.0 |
| 12 | 0.0 | 0.0 | 55.0 | 0.0 | 0.0 | 0.0 | 0.0 | 0.0 | 0.0 | 45.8 | 0.0 | 0.0 |
| 13 | 0.0 | 29.9 | 0.0 | 0.0 | 0.0 | 0.0 | 0.0 | 0.0 | 0.0 | 0.0 | 0.0 | 0.0 |
| 14 | 0.0 | 0.0 | 0.0 | 0.0 | 0.0 | 0.0 | 0.0 | 0.0 | 0.0 | 0.0 | 53.0 | 0.0 |
| 15 | 0.0 | 0.0 | 0.0 | 0.0 | 0.0 | 0.0 | 0.0 | 0.0 | 0.0 | 0.0 | 0.0 | 79.1 |
| 16 | 0.4 | 4.0 | 0.6 | 0.5 | 1.7 | 1.7 | 0.4 | 5.4 | 0.6 | 2.2 | 0.9 | 0.4 |
| *Table 2A ingredient list: 1) sodium tripolyphosphate; 2) sodium aluminosilicate, zeolite structure; 3) sodium carbonate; 4) sodium sulfate; 5) carboxymethyl cellulose; 6) optical brightener powder; 7) milled composition Table 1A column (b); 8) milled composition Table 1A column (d); 9) milled composition Table 1A column (e); 10) milled composition Table 1A column (g); 11) milled composition Table 1A column (h); 12) milled composition | | | | | | | | | | | | |
| # Table 1A column (i); 13) milled composition Table 1A column (j); 14) milled composition Table 1A column (k); 15) milled composition Table 1A column (l); 16) moisture and raw material by products. | | | | | | | | | | | | |

TABLE 2B
Layering Powder Compositions (by mass)

| Ingredients* | (m) | (n) | (o) | (p) | (q) | (r) | (s) | (t) | (u) | (v) | (w) | (x) |
|---|---|---|---|---|---|---|---|---|---|---|---|---|
| 1 | 37.8 | 50.4 | 50.3 | 100 | 48.2 | 57.6 | 45.9 | 48.7 | 48.3 | 76.3 | 67.6 | 0.0 |
| 2 | 20.0 | 8.5 | 6.2 | 0.0 | 0.0 | 0.0 | 0.0 | 0.0 | 0.0 | 8.1 | 0.0 | 0.0 |
| 3 | 0.0 | 34.4 | 26.9 | 0.0 | 20.9 | 42.4 | 32.9 | 0.0 | 0.0 | 15.6 | 0.0 | 23.0 |
| 4 | 0.8 | 0.0 | 0.0 | 0.0 | 8.3 | 0.0 | 12.7 | 0.0 | 23.2 | 0.0 | 32.4 | 0.0 |
| 5 | 0.0 | 0.0 | 14.2 | 0.0 | 10.1 | 0.0 | 0.0 | 25.1 | 0.0 | 0.0 | 0.0 | 18.0 |
| 6 | 7.3 | 0.0 | 0.0 | 0.0 | 0.0 | 0.0 | 0.0 | 0.0 | 0.0 | 0.0 | 0.0 | 0.0 |
| 7 | 9.7 | 0.0 | 0.0 | 0.0 | 0.0 | 0.0 | 0.0 | 0.0 | 0.0 | 0.0 | 0.0 | 0.0 |
| 8 | 18.4 | 0.0 | 0.0 | 0.0 | 0.0 | 0.0 | 0.0 | 0.0 | 0.0 | 0.0 | 0.0 | 0.0 |
| 9 | 1.0 | 0.0 | 0.0 | 0.0 | 0.0 | 0.0 | 0.0 | 0.0 | 0.0 | 0.0 | 0.0 | 0.0 |
| 10 | 1.0 | 0.0 | 0.0 | 0.0 | 0.0 | 0.0 | 0.0 | 0.0 | 0.0 | 0.0 | 0.0 | 0.0 |
| 11 | 2.0 | 0.0 | 0.0 | 0.0 | 0.0 | 0.0 | 0.0 | 0.0 | 0.0 | 0.0 | 0.0 | 0.0 |
| 12 | 0.0 | 0.0 | 0.0 | 0.0 | 0.0 | 0.0 | 8.5 | 0.0 | 0.0 | 0.0 | 0.0 | 0.0 |
| 13 | 0.0 | 6.7 | 0.0 | 0.0 | 0.0 | 0.0 | 0.0 | 0.0 | 0.0 | 0.0 | 0.0 | 59.0 |
| 14 | 0.0 | 0.0 | 0.0 | 0.0 | 12.5 | 0.0 | 0.0 | 0.0 | 0.0 | 0.0 | 0.0 | 0.0 |
| 15 | 0.0 | 0.0 | 2.4 | 0.0 | 0.0 | 0.0 | 0.0 | 0.0 | 0.0 | 0.0 | 0.0 | 0.0 |
| 16 | 0.0 | 0.0 | 0.0 | 0.0 | 0.0 | 0.0 | 0.0 | 26.2 | 28.5 | 0.0 | 0.0 | 0.0 |
| 17 | 2.0 | 0.0 | 0.0 | 0.0 | 0.0 | 0.0 | 0.0 | 0.0 | 0.0 | 0.0 | 0.0 | 0.0 |
| *Table 2B ingredient list: 1) sodium carbonate; 2) sodium sulfate; 3) sodium citrate; 4) MGDA; 5) GLDA; 6) sodium silicate; 7) sodium acrylic-maleic copolymer; 8) sodium alkyl benzene sulfonate; 9) HEDP; 10) EDDS; 11) magnesium sulfate; 12) milled composition Table 1B column (o); 13) milled composition Table 1B column (p); 14) milled composition Table 1B column (r); 15) milled composition Table 1B column (s); 16) milled composition Table | | | | | | | | | | | | |
| # 1B column (v); 17) moisture and raw material by products. | | | | | | | | | | | | |

### Example 3

### Binders

While the binder choice depends on the application, a preferred binder system includes at least one binder component that is capable of undergoing a chemical or physical transformation from a liquid to solid or semi-solid phase. In the case of a chemical transformation, the binder preferentially reacts with a component of the layering powder. Suitable non-reactive binders may be used to the extent that they can be physically adsorbed in the layering structure. Examples of suitable non-reactive binders include, but are not limited to, perfume oils, flavoring oils and nutritional oils.

For detergent applications, suitable active binder materials include, but are not limited to, materials selected from the group consisting of acid surfactant precursors, liquid or molten surfactants or surfactant solutions of anionic, cationic, nonionic or zwitterionic surfactants, liquid or molten polymers, polymer solutions, acidic polymers, silicate solutions, cellulosic solutions or dispersions, molten fatty acids or alcohols, waxes and mixtures thereof. Suitable inert binder materials include, but are not limited to, materials selected from the group consisting of water, salt solutions, sugar solutions and mixtures thereof.

### Example 4

### Process for Making a Layered Granule for Automatic Dishwashing Detergent Using Spatially-Separated Binder and Layering Powder Streams

The seed particle composition in Table 1A column (a), obtained from Procter & Gamble Co., is sieved to a particle size cut of between about 300 and 850 microns, using a Sweco 24″ Vibro-Energy Round Separator. A mass of about 75 kg of the seed particles, with a bulk density of about 1.07 kg/liter is then dosed into a dual-axis counter-rotating paddle mixer (Bella™ B-120XN, available from Dynamic Air, St. Paul, Minn., USA), modified for binder addition using a distributor pipe located below the converging flow zone. The mixer is turned on, with two shafts counter-rotating at about 100 RPM. Each shaft has 14 paddles mounted in 7 pairs per shaft. A liquid mass of about 0.6 kg of linear alcohol alkoxylate, heated to obtain a viscosity of about 40 cp, is added via pressure spray nozzle into the top of the mixer at a rate of about 200 lbs/hr, so as to form atomized droplets and then contact said droplets with the particles in the center of the mixer, where the seed particles are fluidized. An atomized spray of sodium polyacrylate polymer binder solution of about 30 wt % solids is then started through nozzles mounted on the top-center of the mixer so as to contact binder droplets with the fluidized seed particles in the center of the mixer. The polymer solution is sprayed on at a rate of about 75 lbs/hr for about 6 minutes. Concurrently, the layering powder of Table 2A column (a) is added into the top of the mixer, split equally through two ingress ports located at diagonal corners of the mixer, directed over positions of downward paddle trajectory nearest to the end walls of the mixer, at a rate of about 900 lbs/hr for 6 minutes. Also concurrently, a sodium silicate binder solution of about 34 wt % solids is added through the distributor bar directing flow upwards into the converging flow zone through four holes of about 2 mm diameter straddling the center three paddle positions. The sodium silicate solution is added at a rate of about 175 lbs/hr for about 5½ minutes. The entire process is allowed to take place over a 6 to 7 minute residence time in the mixer prior to discharge into a second mixer, Bella™ B-200XN with similar modifications for binder addition. The process is then repeated in the second mixer, using the product of the first mixer as seeds, for a similar residence time and including all layering ingredients except the linear alkoxylate, with addition rates for layering powder, silicate and polymer solutions of about 1450, 260, and 110 lbs/hr, respectively.

The resulting batch is discharged, screened to remove any oversize (>1.2 mm) and treated in a fluid bed with ambient temperature air and a superficial air velocity of about 0.8 m/s for 3 to 7 minutes. The product yield is about 90% accepts; the remainder is treated by milling and recycled either as seeds or layering powder. The cumulative layering process residence time is about 14 minutes, not including the fluid bed treatment. The free moisture in the binder solutions is substantially reacted with the phosphate and carbonate constituents of the layering powder during the layering process, leading to an equivalent conversion of about 80% sodium tripolyphosphate hexahydrate and about 50% sodium carbonate monohydrate. No further product drying is required. The product growth factor is about 2.8 times the amount of initial seed granules. The layering rate is about 20 mass % per minute. The product particle size is characterized by D₅₀=630 microns, span=1.3, and D₃₀=540 microns. The product Relative Jamming Onset is about 6.9 particles.

### Example 5

### Process for Making a Layered Granule for Automatic Dishwashing Detergent Using Temporally Separated Binder and Layering Powder Streams

The seed particle composition in Table 1A column (a), obtained from Procter & Gamble Co., is sieved to a particle size cut of between about 300 and 850 microns, using a Sweco 24″ Vibro-Energy Round Separator. A mass about 320 g of the seed particles, with a bulk density of about 1.07 kg/liter is then dosed into a high shear food processing-type kitchen mixer (Robot Coupe, model R302V). The mixer is turned on at the low setting so as to create a centrifugal “rope flow” of seed particles rotating in a centrifugal flow pattern against the wall of the mixer. About 4.6 grams of liquid linear alcohol alkoxylate is dosed into the mixer via a syringe through the top inlet such that the liquid stream contacts the seed particles at an approximately perpendicular angle to the surface of the flow pattern. Sequentially, about 60 grams of layering powder composition given in Table 2A column (a) is added by through the top of the mixer. Sequentially, about 60 grams of a sodium silicate binder solution of about 36 wt % solids is added through the top of the mixer, at an approximately perpendicular angle to the surface of the flow pattern. Sequentially, about 145 grams of layering powder composition given in Table 2A column (a) is added by through the top of the mixer. Sequentially, about 27 grams of a sodium polyacrylate polymer binder solution of about 26 wt % solids is added through the top of the mixer, at an approximately perpendicular angle to the surface of the flow pattern. Finally, about 100 grams of layering powder composition given in Table 2A column (a) is added through the top of the mixer. During the batch process, the mixer speed is gradually increased to keep the material moving in a centrifugal flow pattern against the wall of the mixer.

### Example 6

### Process for Making an Effervescent Layered Granule for Medium-Duty Laundry Detergent Using Temporally Separated Binder and Layering Powder Streams

Seed particles are obtained by classifying granular Sodium Bisulfate using screens and selecting the cut between 500 and 1000 microns. Layering powder of Table 2A column (c) is used. The binder is prepared by mixing about 85% linear alkyl benzene sulfonic acid (HLAS) with about 15% molten Tallow Alcohol Ethoxylate (TAE80) at a mixture temperature of about 60° C. The homogeneous binder mix is then kept at about 60° C.

A mass of 203 grams of the seed material is loaded into a Food Processor Model FP370 and the mixer turned on to speed setting #2 to induce a centrifugal flow pattern in the mixer. A series of eight sequential layering steps are then performed, alternately adding about 15 grams of binder and about 35 to 45 grams of layering powder, adding more binder, more layering powder, etc., until the product composition is built up in layers surrounding the bisulfate seeds.

The binder is converted to a solid phase by a combination of chemical reaction of the HLAS binder with Sodium Carbonate in the layering powder and congealing of the molten TAE80. Without being bound by theory, it is thought that the blended binder system extends the capability of such processing to lower levels of excess sodium carbonate in the layering powder. In addition, the substantially non-aqueous process enables the formation of a composite particle with an acidic core structure (sodium bisulfate) and alkaline layers, such that, when added to water, the particulate effervesces.

### Example 7

### Process for Making a Layered Heavy-Duty Detergent Granule Containing Perfume Microcapsules Using Temporally Separated Binder and Layering Powder Streams

Seed particles are obtained by first preparing the intermediate granular composition provided in Table 1A column (k) by a spray-drying process. The resultant spray-dried granules are classified by screening, with the seeds taken from the size cut between 425 microns and 850 microns. The resultant seeds have a bulk density of about 300 grams/liter, with a porous microstructure.

A mass of 200 grams of the seed material is loaded into a Braun CombiMax 600 Food Processor, type 3205 with blade impeller and the mixer turned on to a speed sufficient to induce a centrifugal flow pattern in the mixer, for example, speed setting #4. Nine grams of an aqueous slurry of perfume microcapsules prepared in accordance with U.S. Pat. No. 4,100,103, containing about 30 wt % active perfume oil, is then added by syringe such that the stream of the slurry contacts the flow of porous seeds, embedding the microcapsules into the porous particle structure.

The layering powder composition is provided in Table 2A column (k). Two separate binders are used: 1) an acid surfactant precursor such as alkyl benzene sulfonic acid (HLAS) and/or alkyl 3-elthoxysulfonic acid (HAE3S), and 2) a sodium polyacrylate solution of about 30 wt % solids. The acid surfactant precursor converts to its sodium salt on contact with fine sodium carbonate in the layering powder. The polyacrylate solution also solidifies by hydration of sodium carbonate.

A series of 6 sequential layering steps are then performed, alternately adding about 11 grams of acid surfactant precursor binder by syringe, about 45 grams of layering powder by teaspoon, and then about 1 gram of polyacrylate solution by syringe, all delivered sequentially through the top of the mixer, contacting the particulate flow in the mixer. Then the total mass is discharged and classified using sieves, 330 grams are taken from the size cut between 300 microns and 1180 microns and returned to the mixer.

The layering process is then repeated, with a series 6 sequential layering steps, alternately adding about 10 grams of acid surfactant precursor binder by syringe, about 50 grams of layering powder by teaspoon, and then about 1 gram of polyacrylate solution by syringe, all delivered sequentially through the top of the mixer, contacting the particulate flow in the mixer.

The material is discharged from the mixer and classified using screens to obtain a product with a particle size between about 300 microns and 1180 microns. The resulting bulk density of the product is about 800 grams/liter.

### Example 8

### Process for Making a Layered Heavy-Duty Detergent Granule Using Spatially Separated Binder and Layering Powder Streams and Spray-Dried Seeds

The seed particle composition of Table 1A column (e) is prepared by spray-drying followed by classification between 300 micron and 850 micron screens, using a Sweco 24″ Vibro-Energy Round Separator. A layering powder composition of Table 2A column (e) is prepared using a Netzsch CUM-150 pin mill to grind the fine tails of the above spray-dried material as well as sodium carbonate to a median particle size of about 20 microns. Two separate binders are used: linear alkyl benzene sulfonic acid (HLAS), and an aqueous solution of acrylic-maleic copolymer with about 30 wt % solids.

A mass of about 8 kg of the seed particles, with a bulk density of about 0.45 kg/liter is then charged into a dual-axis counter-rotating paddle mixer (Bella™ B-32XN). The mixer is turned on, with two shafts counter-rotating at about 160 RPM. Each shaft has 22 paddles mounted in 11 pairs per shaft. An atomized spray of sodium polyacrylate polymer binder solution of about 30 wt % solids is added through the top of the mixer so as to contact binder droplets with the particles in the center of the mixer, where the seed particles are fluidized. The HLAS binder is added through the bottom of the mixer by use of a 4-holed distributor bar, directing flow upwards into the converging flow zone, straddling the center three paddle positions. The layering powder is added into the top of the mixer, split through two ingress ports located at diagonal corners of the mixer, directed over positions of downward paddle trajectory nearest to the end walls of the mixer. The binders and layering powers are added concurrently as per the “Step 1” section of Table 3, Addition Schedule. After the Step 1 schedule is complete, a mass of about 11.55 kg of the Step 1 particulate product is charged into the same mixer for use as seeds, and the process is repeated according to Table 3, Step 2. The resulting batch is discharged, screened to remove any oversize (>1.2 mm) and treated in a fluid bed with ambient temperature air and a superficial air velocity of about 0.8 m/s for about 4 minutes. The binders are substantially converted to solid phases within the layering process and no further drying is required. The product yield is about 90% accepts. The bulk density is about 0.82 kg/liter. This product is then further layered with a small amount of perfume oil binder (about 0.2 mass %) and zeolite layering powder (about 0.8 mass %) according to Table 3, Step 3. The perfume layering step is done using the same mixer using about 20 kg of the treated Step 2 product as seeds and a finely atomized spray of perfume added through the top of the mixer so as to contact spray droplets with the particles in the center of the mixer, where the seed particles are fluidized. The total mass-based growth factor of the product relative to initial seeds is about 5.5. The Relative Jamming Onset is about 7.3 particles.

In a manufacturing production scenario, this process may be scaled-up to run with two mixers arranged in series, the second mixer containing about two times the working volume of the first. In this scenario, the particulate product of Step 1 is discharged from mixer 1 into mixer 2 for use as seeds in Step 2. The Step 2 process may be completed in substantially the same time as Step 1, such that the two mixers can be operated in a synchronized batch schedule with minimal idle time. To maintain similar batch times, the Step 2 feed rates of binder and layering powder may be scaled up in proportion to the batch size. Under this production scenario, the Layering Rate can be about 60 mass % per minute or even greater. The Yield Rate can be greater than 50 mass % per minute.

TABLE 3
Addition Schedule for Example 8

| | Addition rate | Start time | Stop time |
|---|---|---|---|
| | (kg/min) | (mm:ss) | (mm:ss) |
| Step 1 | | | |
| Start mixer, 160 RPM | | 0:00 | 4:10 (discharge) |
| Polymer solution | 0.25 | 0:05 | 0:40 |
| HLAS binder | 1.00 | 0:20 | 2:49 |
| Layering powder | 3.00 | 0:30 | 3:54 |
| Polymer solution | 0.25 | 2:26 | 4:00 |
| Step 2 | | | |
| Start mixer, 160 RPM | | 0.00 | 3:50 (discharge) |
| HLAS binder | 1.00 | 0:05 | 2:19 |
| Layering powder | 3.00 | 0:05 | 3:33 |
| Polymer solution | 0.25 | 2:24 | 3:38 |
| Step 3 | | | |
| Start mixer, 160 RPM | | 0.00 | 1:10 (discharge) |
| Perfume oil binder | 0.10 | 0:05 | 0.33 |
| Zeolite layering powder | 0.40 | 0:10 | 0.38 |

### Example 9

### Process for Making a Layered Heavy-Duty Detergent Granule Using Separated Binder and Layering Powder Streams and Granular Seeds

This example builds layer mass sequentially over three steps, each conducted as batch in a pilot-scale paddle mixer. The granular seed particle composition of Table 1A column (j) is prepared by a mechanical agglomeration process followed by classification of the granules between 380 micron and 850 micron screens, using a Sweco 24″ Vibro-Energy Round Separator. A layering powder composition is prepared by blending 2:1 mass ratio of micronized soda ash and zeolite A powders. Two separate binders are used: linear alkyl benzene sulfonic acid (HLAS) and an aqueous solution of sodium polyacrylate polymer with about 30 wt % solids.

A mass of about 10 kg of the seed particles, with a bulk density of about 0.8 kg/liter is then charged into a dual-axis counter-rotating paddle mixer (Bella™ B-20XE). The mixer is turned on, with two shafts counter-rotating at about 120 RPM. Each shaft has 14 paddles mounted in 7 pairs per shaft. The binder is added in sequential stages. First, an atomized spray of heated HLAS binder, about 60 C, with a viscosity of about 150 cp is added through the top of the mixer so as to contact binder droplets with the fluidized seed particles in the center of the mixer. Second, the polymer solution binder is also sprayed from the top of the mixer, onto the same center fluidized zone, using a separate nozzle. Concurrent with the binder sprays, layering powder is added into the top of the mixer, through one ingress port located over a corner of the mixer top, dropping onto an outside (downward moving) paddle position. The binders and layering powers are added as per the “Step 1” section of Table 4, Recipe for Example 9. After the Step 1 schedule is complete, a mass of about 11.16 kg of the Step 1 particulate product is charged into the same mixer, and the process is repeated according to Table 4, Step 2. After the Step 2 schedule is complete, a mass of about 11.65 kg of the Step 2 particulate product is charged into the same mixer, and the process is repeated according to Table 4, Step 3. Depending on the stage of the process, the coalescence Stokes Number ranges between about 7 and 9, and the layering Stokes Number ranges between 0.5 and 0.7. The resulting batch is discharged, screened to remove any oversize (>1.2 mm). The product yield is about 95% accepts. The bulk density is about 950 grams/liter. The mass-based growth factor of the product relative to seeds is about 5.3. The Relative Jamming Onset is about 6.1 particles. The median particle aspect ratio is about 1.22.

TABLE 4
Recipe for Example 9 (in grams)

| | | Step 1 | Step 2 | Step 3 |
|---|---|---|---|---|
| Load seeds (step 1) | | 10000 | | |
| Load partial previous product (steps | | | 11158 | 11646 |
| 1→2, 2→3) | | | | |
| a) | HLAS binder | 1512 | 1451 | 1306 |
| | Layering powder | 4424 | 4247 | 3822 |
| | loss on reaction (CO₂) | −111 | −107 | −96 |
| b) | Polymer solution | 389 | 374 | 336 |
| | Layering powder | 2382 | 2287 | 2058 |

| | | | |
|---|---|---|---|
| Total | 18596 | 19410 | 19073 |

### Example 10

### Process for Making a Layered Heavy-Duty Detergent Granule Using Separated Binder and Layering Powder Streams and Sulfate Seeds

This example builds layer mass sequentially over three steps, each conducted as batch in a 20 liter pilot-scale ploughshare mixer. A suitable ploughshare mixer can be obtained from Lodige GMBH, The seed particle is obtained in the form of coarse granular sodium sulfate with a median particle size of about 600 um. A layering powder composition of Table 2A column (g) is prepared using a Netzsch CUM-150 pin mill to obtain a median particle size of about 20 microns. A small amount of zeolite powder is used to supplement the layering powder. The binder is linear alkyl benzene sulfonic acid (HLAS)

The product is made over a series of three batch steps, as per Table 5, Recipe for Example 10, using a medium shear ploughshare mixer (Lodige M-20-G Lab Plow Mixer, with a ploughshare agitator radial sweep of about 0.15 meters). The mixer is turned on, with main agitator shaft rotating at about 175 RPM and the chopper running at about 3000 RPM. A stream of heated HLAS binder (about 60° C.) with a viscosity of about 150 cp is added through an addition pipe below the chopper. The layering powder is added into the top of the mixer above the chopper location. The coalescence Stokes number, St_{coalescence}, is about 17 and the layering Stokes number, St_{layering}, is about 1.1.

The resulting batch is discharged, screened to remove any oversize (>1.4 mm). The product yield is about 95% accepts. The bulk density is about 1.05 grams/liter. The mass-based growth factor of the product relative to seeds is about 4.5. The D₃₀ particle size is about 895 um, and the Relative Jamming Onset is about 5.8 particles.

TABLE 5
Recipe for Example 10 (in grams)

| | Step 1 | Step 2 | Step 3 |
|---|---|---|---|
| Load seeds (step 1) | 3700 | | |
| Load partial previous product (steps | | 3147 | 3792 |
| 1→2, 2→3) | | | |
| HLAS binder | 730 | 803 | 522 |
| Layering powder | 1345 | 1942 | 1263 |
| Loss on reaction (CO₂) | −54 | −59 | −38 |
| Zeolite powder | 100 | 100 | 200 |
| Total | 5821 | 5933 | 5739 |

### Example 11

### Process for Making a Layered Heavy-Duty Detergent Granule Using Temporally-Separated Binder and Layering Powder Streams and a Mixture of Granular Seeds

The seed particle compositions given in Table 1A column (I) and Table 1B column (m) are prepared by spray drying and mechanical agglomeration processes, respectively, followed by classification between 425 micron and 1400 micron screens. A layering powder composition is prepared according to Table 2A column (1). A binder mix of linear alkyl benzene sulfonic acid (HLAS) and Ethoxylated Hexamethylene Diamine Quat (EHDQ) is prepared using a mass ratio of about 86% HLAS and 14% of the EHDQ. The binder mixture is heated to about 60 C, with a viscosity of about 150 cp.

A mass of about 0.28 kg of the seed particles, consisting of a mass ratio of about 25% granules of Table 1A column (1) and 75% granules of Table 1B column (m), with an combined bulk density of about 0.8 kg/liter, is loaded into a Kenwood Food Processor Model FP370 and the mixer turned on to speed setting #2 to induce a centrifugal flow pattern in the mixer. A series of four sequential layering steps are then performed, alternately adding about 15 grams of binder drop-wise via a syringe, contacting the seed particles in the mixer, followed by about 15 to 25 grams of layering powder, also added through the top of the mixer, adding more binder, more layering powder, etc., until the product composition is built up in layers surrounding the seed particles.

### Example 12

### Determination of Jamming Onsets

In this example, the details are provided for the determination of the Relative Jamming Onset and Absolute Jamming Onset for the layered granule of Example 9.

First, the 30^{th} percentile particle size (D₃₀) is measured according to Method 5, “Flowable Particle Mass Based Cumulative Particle Size Distribution Test.” The 30^{th} cumulative mass % lies between 600 um and and 850 um., as per Table 6, “Particle Size Data.” Interpolation of the 30^{th} percentile relative to the Log(size) data results in a Log(D₃₀) of 2.8542 and a D₃₀ of 715 um.

TABLE 6
Particle size data for Example 12.

| Screen | Mass % on | Cumulative mass % | |
|---|---|---|---|
| size (um) | screen | finer | Log (size) |
| 2360 | 0.00 | 100.00 | 3.3729 |
| 1700 | 0.00 | 100.00 | 3.2304 |
| 1180 | 1.14 | 98.86 | 3.0719 |
| 850 | 42.12 | 56.74 | 2.9294 |
| 600 | 53.79 | 2.95 | 2.7782 |
| 425 | 2.34 | 0.60 | 2.6284 |
| 300 | 0.46 | 0.14 | 2.4771 |
| 212 | 0.10 | 0.04 | 2.3263 |
| 150 | 0.03 | 0.01 | 2.1761 |
| pan | 0.01 | 0.00 | |

The Relative and Absolute Jamming Onsets are determined in accordance with Method 6, “Jamming Onset.” Data obtained from the test are provided in Table 7, “Jamming Onset Data.” To obtain the dimensionless Relative Jamming Onset, the D₃₀ particle size is converted to the same units as the orifice dimension. The required 25 mass % discharge falls between the dimensionless orifice size (b) of 5.59 and 6.99. Interpolation relative to the mass % discharged data results in a measured Relative Jamming Onset of 6.07 particles and an Absolute Jamming Onset of 4.34 millimeters.

TABLE 7
Jamming Onset data or Example 12 (D₃₀ = 715 um = 0.715 mm).

| | | | | | |
|---|---|---|---|---|---|
| Orifice | B (mm) | 3.5 | 4 | 5 | 6 |
| | b = B/D₃₀ | 4.90 | 5.59 | 6.99 | 8.39 |
| Trial 1 | Load (g) | 120.2 | 120.7 | 120.7 | 120.7 |
| | discharge (g) | 0.1 | 1.8 | 81.6 | 83.6 |
| | % discharge | 0.08% | 1.49% | 67.61% | 69.26% |
| Trial 2 | Load (g) | 120.1 | 120.7 | 120.7 | 120.7 |
| | discharge (g) | 0.23 | 0.05 | 81.9 | 83.4 |
| | % discharge | 0.19% | 0.04% | 67.85% | 69.10% |
| Trial 3 | Load (g) | 120.1 | 120.7 | 120.7 | 120.7 |
| | discharge (g) | 0.05 | 8.4 | 81.55 | 82.33 |
| | % discharge | 0.04% | 6.96% | 67.56% | 68.21% |
| Average | % discharge | 0.11% | 2.83% | 67.67% | 68.97% |

### Example 13

### Process for Making a Layered Granule for Automatic Dishwashing Detergent Using Spatially-Separated Binder and Layering Powder Streams

The seed particle composition in Table 1B column (p), made by a spray-drying process, is sieved to a particle size cut of between about 300 and 850 microns, using a Sweco 24″ Vibro-Energy Round Separator. A mass of about 350 kg of the seed particles, with a bulk density of about 0.6 kg/liter is then dosed into a dual-axis counter-rotating paddle mixer (Bella™ B-1000XN), modified for binder addition using a distributor pipe located below the converging flow zone. The mixer is turned on, with two shafts counter-rotating at about 45 RPM. Each shaft has 14 paddles mounted in 7 pairs per shaft. A liquid mass of about 20 kg of linear alcohol alkoxylate, heated to obtain a viscosity of about 40 cp, is added via pressure spray nozzle into the top of the mixer at a rate of about 10 kg/minute so as to form atomized droplets and then contact said droplets with the fluidized seed particles in the center of the mixer. After the addition of the linear alcohol alkoxylate, a sequential combination of binders and layering powders is added to create an inner layer of comparatively hygroscopic chemistry surrounded by an outer layer of less hygroscopic material. The total layering time after the alkoxylate addition is about 8 minutes. The sequential layering powder addition includes two layering powders. A first layering powder of Table 2B column (x), added into the top of the mixer, split equally through two ingress ports located at diagonal corners of the mixer, directed over positions of downward paddle trajectory nearest to the end walls of the mixer, at a rate of about 45 kg/minute for 5 minutes. After the addition of the first layering powder is complete, a second layering powder of Table 2B column (p) is added through the same ingress ports at a rate of about 40 kg/minute for 3 minutes and 15 seconds.

Concurrent with start of the layering powder additions, a sodium silicate binder solution of about 41 wt % solids is added through the bottom of the mixer by use of a 4-holed distributor bar, directing flow upwards into the converging flow zone, straddling the center three paddle positions. The sodium silicate solution is added at a rate of about 11 kg/minute for about 8 minutes. Concurrent with the addition of the sodium silicate binder, an atomized spray of sodium polyacrylate polymer binder solution of about 32 wt % solids is added through nozzles mounted on the top-center of the mixer so as to contact binder droplets with the particles in the center of the mixer, where the seed particles are fluidized. The polymer solution is sprayed on at a rate of about 3 kg/minute for about 8 minutes.

The resulting batch is discharged, screened to remove any oversize (>1.2 mm) and dried in a fluid bed with an air inlet temperature of about 130 C and an air flow of about 260 kg/minute for about 10 minutes. The product yield is about 90% accepts; the remainder is treated by milling and recycled either as seeds or layering powder.

The Relative Jamming Onset of the accepted particulate is about 7.2 particles, and the product Rapid Stability Relative Jamming Onset is about 8.0 particles.

### Example 14

### Continuous Process for Making a Layered Granule for Automatic Dishwashing Detergent

The seed particle composition in Table 1B column (s), made by a mechanical agglomeration process, is continuously sieved to a particle size cut of between about 420 and 1000 microns, using a multi-deck Mogensen Sizer®. The tailings from the sieving process are suitably recycled back to the agglomeration process. The classified seed material is added continuously into the primary inlet of a Lodige KM-600 mixer at a rate of about 650 kg/hour. The KM-600 mixer is fitted with ploughshare mixing elements rotating at a tip-speed of about 2 meters/second. Two high-speed choppers are located between plough positions along the axial direction of the mixer. A 41% aqueous solution of sodium silicate is added continuously to the KM-600 mixer through two pipe inlets beneath the chopper blades. The combined flow rate of the silicate solution is about 75 kg/hour. Sodium Carbonate Anhydrous powder is micronized using a Netzsch-Condux CUM-150 pin-mill to a form a fine layering powder, and then added continuously into the mixer at two locations above the choppers. The layering powder is added at a combined rate of about 275 kg/hour. The total throughput rate of the continuous layering process is about 1 metric ton/hour. The water in the silicate solution is substantially hydrated by the sodium carbonate layering powder. No further drying is required.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the same term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A process for producing particles comprising:
a.) layering a mass of seeds, said seeds having:
(i) a median particle diameter of from about 150 microns to about 1700 microns;
(ii) optionally a size distribution span of from about 1.0 to about 2.0;
(iii) a seed bulk density of from about 50 grams per liter to about 2000 grams per liter; and
(iv) optionally a median particle aspect ratio of from about 1 to about 2;
said layering process comprising independently contacting said mass of seeds with a binder having a viscosity of from about 0.5 cp to about 4000 cp and a layering powder having a median particle size from about 1 micron to about 100 microns, and optionally repeating said layering step;
b.) optionally, conducting said process at a Layering Stokes Number of from greater than 0 to about 10;
c.) optionally, conducting said process at a Coalescence Stokes Number of at least 0.5;
d.) optionally, treating said particles to remove any materials that would result in said particles having a Relative Jamming Onset of greater than about 14.

2. The process of claim 1 wherein said layering process of independently contacting said mass of seeds with a binder and a layering powder is selected from the processes of simultaneously contacting said mass of seeds with independent streams of said binder and said layering powder, contacting said mass of seeds with a stream of said binder and then contacting said mass of seeds with a stream of said layering powder, contacting said mass of seeds with a stream of said layering powder and then contacting said mass of seeds with a stream of said binder and, when more than one layer is required, optionally combinations thereof.

3. The process of claim 1 wherein said layering step is repeated a sufficient number of times to increase the product mass by a factor of more than about two compared to the initial seed mass.

4. The process of claim 1 wherein the Layering Rate is more than about 5 mass % per minute.

5. The process of claim 1 wherein the mass of seeds and layering powder are introduced into the process at separate times but at substantially identical physical locations.

6. A process for producing particles comprising:
a.) layering a mass of seeds with a binder comprising a liquid and a layering powder said process comprising independently contacting said mass of seeds with said binder and said layering powder, said processing being conducted at a layering Stokes Number of from greater than 0 to about 10 and a Coalescence Stokes Number of at least 0.5; and
b.) optionally layering said mass of seeds one or more times in accordance with the process parameters of a.) above; and
c.) treating said particles to remove any materials that would result in said particles having a Relative Jamming Onset of greater than about 14.

7. The process of claim 6 wherein said layering process of independently contacting said mass of seeds with a binder and a layering powder is selected from the processes of simultaneously contacting a mass of seeds with independent streams of said binder and said layering powder; contacting said mass of seeds in a first location with a stream of said binder and then contacting said seed-binder mixture with a stream of said layering powder in a second location; contacting a mass of seeds with a stream of said layering powder in a first location and then contacting said seed-powder mixture with a stream of said binder in a second location or combination thereof.

8. The process of claim 6 wherein said layering step is repeated a sufficient number of times to increase the product mass by a factor of more than about two compared to the initial seed mass.

9. The process of claim 6 wherein the Layering Rate is more than about 5 mass % per minute.

10. The process of claim 6 wherein the mass of seeds and layering powder are introduced into the process at separate times but at substantially identical physical locations.

11. A particulate produced by the process of claim 1.

12. A particulate comprising particles, said particles comprising at least one seed and at least one layer that at least partially coats said seed, said particulate having a Relative Jamming Onset of from about 2 to about 14 and a median particle size of from about 250 microns to about 4,000 microns.

13. The particulate of claim 12, said particulate having a median particle size of from about 600 microns to about 750 microns.

14. The particulate of claim 12, said particulate having a bulk density of from about 350 grams/liter to about 2000 grams/liter.

15. The particulate of claim 12, said particulate having a median particle aspect ratio from about 1.0 to about 1.4.

16. The particulate of claim 12, where at least one of said one or more seeds is selected from the group consisting of hollow particles, seeds comprising pores, encapsulates, seeds comprising pores containing an active material and mixtures thereof.

17. The particulate of claim 12, where in said seed has:
(i) a median particle diameter of from about 150 microns to about 1700 microns;
(ii) optionally a size distribution span of from about 1.0 to about 2.0;
(iii) a seed bulk density of from about 50 grams per liter to about 2000 grams per liter; and
(iv) optionally, a median particle aspect ratio of from about 1 to about 2.

18. The particulate of claim 12, said particulate having a Rapid Stability Relative Jamming Onset of from about 2 to about 18.

19. A finished product comprising the particulate according to claim 12.

20. The finished product according to claim 19, said finished product having a Relative Jamming Onset of from about 2 to about 14.

21. A finished product comprising the particulate according to claim 17.

22. The finished product according to claim 21, said finished product having a Relative Jamming Onset of from about 2 to about 14.

23. A finished product comprising the particulate according to claim 16, said finished product having a Relative Jamming Onset of from about 2 to about 14.

24. A finished product comprising the particulate according to claim 18, said finished product having a Rapid Stability Relative Jamming Onset of from about 2 to about 18.

25. A method of treating and/or cleaning, said method comprising:
a.) optionally washing and/or rinsing a situs;
b.) contacting at least a portion of a situs with a particulate according to claim 12 and/or a finished product according to claim 19; and
c.) then optionally washing and/or rinsing said situs or said portion of said situs.

26. A packaged product comprising the finished product of claim 20, said packaged product containing the product of claim 20, said packaged product comprising a product dosing orifice having a Critical Gap Dimension,that is from about greater than the Absolute Jamming Onset of said finished product but less than four times, said Absolute Jamming Onset.

27. A packaged product comprising a finished product comprising the particulate of claim 11, said packaged product comprising a product dosing orifice having a Critical Gap Dimension that is from about 2 mm to about 11 mm.

28. The packaged product of claim 26, said packaged product contained in a package that is at least partially transparent.

29. The packaged product of claim 27, said packaged product contained in a package that is at least partially transparent.

30. A process for producing particles comprising:
a.) layering a mass of seeds, said seeds having:
(i) a median particle diameter of from about 150 microns to about 1700 microns;
(ii) optionally a size distribution span of from about 1.0 to about 2.0; and
(iii) optionally a median particle aspect ratio of from about 1 to about 2;
said layering process comprising independently contacting said mass of seeds with a binder comprising a liquid and a layering powder having a median particle size from about 1 micron to about 100 microns, and optionally repeating said layering step;
b.) optionally, treating said particles to remove any materials that would result in said particles having a Relative Jamming Onset of greater than about 14.

31. A process for producing a particulate comprising contacting a particle and a binder comprising a liquid in a counter-rotating dual-axis paddle mixer, wherein said binder is introduced into said mixer through an ingress located at the bottom of said dual-axis paddle mixer.

32. The process of claim 31 wherein said binder is introduced such that said binder is directed upward into the converging flow zone between the counter-rotating paddle axes.

33. The process of claim 31 wherein said counter-rotating dual-axis paddle mixer has a converging flow zone between the counter-rotating paddle axes and the swept volumes of said counter-rotating paddle axes do not overlap within the converging flow zone and said binder is directed into the gap between the swept volumes of said counter-rotating paddle axes.

34. The process of claim 31, wherein said binder has a viscosity of from about 1 cp to about 100000 cp.

35. The process of claim 31, wherein said ingress comprises a distributor pipe located below the converging flow zone of the counter-rotating paddle axes said distributor pipe comprising one or more holes.

36. The process of claim 1, wherein said independently contacting said mass of seeds with a binder comprising a liquid and a layering powder comprises introducing said binder into a counter-rotating dual-axis paddle mixer having a converging flow zone between the counter-rotating paddle axes such that said binder is directed upward into the converging flow zone between said counter-rotating paddle axes.

37. The process of claim 1, wherein said independently contacting said mass of seeds with a binder comprising a liquid and a layering powder comprises introducing said binder into a counter-rotating dual-axis paddle mixer having a converging flow zone between the counter-rotating paddle axes wherein the swept volumes of said counter-rotating paddle axes do not overlap within the converging flow zone and said binder is directed into the gap between the swept volumes of said counter-rotating paddle axes.

38. The process of claim 1, wherein said independently contacting said mass of seeds with a binder comprising a liquid and a layering powder comprises introducing said layering powder into a counter-rotating dual-axis paddle mixer having multiple layering powder ingress locations and mixing paddles having a downward trajectory, such that said layering powder is introduced in more than one of said locations in the downward trajectory of the mixing paddles.

39. The process of claim 1 wherein the Product Yield is greater than about 80 mass %.

40. The process of claim 1 wherein the Yield Rate is greater than about 4 mass % per minute.

41. The particulate of claim 12 wherein the particulate effervesces on contact with water.

42. The particulate of claim 12 wherein said particulate is:
a.) an industrial chemical;
b.) an edible food, instant beverage mix, drug or nutriceutical;
c.) a pet food and/or pet care particulate; or
d.) a detergent, fabric treatment, personal cleaning, hair care and/or fertilizer particulate.

43. The finished product of claim 19 wherein said finished product is:
a.) an industrial chemical;
b.) an edible food, instant beverage mix, drug or nutriceutical;
c.) a pet food and/or pet care product; or
d.) a detergent, fabric treatment, personal cleaning, hair care and/or fertilizer product.
